SEQUENCE LISTING (18 sequences)

SEQ ID NO: 1 - DNA, 21 bp, Artificial Sequence
  Gpam, left primer
gaggcaaggacatttatgtgg

SEQ ID NO: 2 - DNA, 20 bp, Artificial Sequence
  Gpam, right primer
ggtgctttcacaatcactcg

SEQ ID NO: 3 - DNA, 18 bp, Artificial Sequence
  Cebpa, left primer
ctggctctgggtctggaa

SEQ ID NO: 4 - DNA, 19 bp, Artificial Sequence
  Cebpa, right primer
agccacaggggtgtgtgta

SEQ ID NO: 5 - DNA, 19 bp, Artificial Sequence
  Pparg, left primer
ctctcagctgttcgccaag

SEQ ID NO: 6 - DNA, 20 bp, Artificial Sequence
  Pparg, right primer
cacgtgctctgtgacgatct

SEQ ID NO: 7 - DNA, 19 bp, Artificial Sequence
  Fabp4, left primer
gcacggtctctctgcaatc

SEQ ID NO: 8 - DNA, 19 bp, Artificial Sequence
  Fabp4, right primer
acaatcaatcagcgcagga

SEQ ID NO: 9 - DNA, 22 bp, Artificial Sequence
  Ucpl, left primer
ccagtggatgtggtaaaaacaa

SEQ ID NO: 10 - DNA, 19 bp, Artificial Sequence
  Ucpl, right primer
cacagcttggtacgcttgg

SEQ ID NO: 11 - DNA, 18 bp, Artificial Sequence
  Agpat2, left primer
ttcccacctcaagcctgt

SEQ ID NO: 12 - DNA, 18 bp, Artificial Sequence
  Agpat2, right primer
tgccttgtggtcttgtgg

SEQ ID NO: 13 - DNA, 19 bp, Artificial Sequence
  Plin, left primer
ctccggcctttcctctcta

SEQ ID NO: 14 - DNA, 18 bp, Artificial Sequence
  Plin, right primer
gggggagtgatgacatgg

SEQ ID NO: 15 - DNA, 19 bp, Artificial Sequence
  Ube23NORM, left primer
ttagtgatttggcccgtga

SEQ ID NO: 16 - DNA, 19 bp, Artificial Sequence
  Ube23NORM, right primer
tggcttgccaatgaaacat

SEQ ID NO: 17 - DNA, 20 bp, Artificial Sequence
  Wbp11NORM, left primer
gagcaatgtccactgtcagg

SEQ ID NO: 18 - DNA, 18 bp, Artificial Sequence
  Wbp11NORM, right primer
atcccagcaggcaaacat

(19) United States
(12) Patent Application Publication
(10) Pub. No.: US 2011/0244059 A1
(43) Pub. Date: Oct. 6, 2011

(54) INHIBITING OBESITY PROGRESSION BY INHIBITING ADIPOCYTE DIFFERENTIATION WITH A PRE-ADIPOCYTE AUTOPHAGY INHIBITOR

(75) Inventor: Shengkan Jin, Belle Mead, NJ (US)

(73) Assignee: The University of Medicine and Dentistry of New Jersey

(21) Appl. No.: 13/059,994

(22) PCT Filed: Aug. 20, 2009
(86) PCT No.: PCT/US09/54464

§ 371 Date: May 11, 2011

Related U.S. Application Data
(60) Provisional application No. 61/189,628, filed on Aug. 20, 2008.

(51) Int. Cl. A61K 33/00; A61K 31/4706; A61K 31/711; A61K 31/7105; A61K 31/713; A61K 31/7088; A61K 31/5415; A61K 31/55; A61K 31/573; A61K 31/57; A61P 3/10; A61P 3/04; A61P 3/00; A61P 9/00; A61P 3/06; A61P 35/00; A61P 1/16; A61P 11/06; A61P 25/20; A61P 19/02; A61P 19/06

(52) U.S. Cl. 424/722; 514/313; 514/44 R; 514/44 A; 514/226.2; 514/217; 514/171; 514/170

(57) ABSTRACT

The present invention relates to methods of mitigating, preventing or treating weight gain or obesity in patients by administering one or more autophagy inhibitors, thereby, preventing the differentiation process of pre-adipocyte cells into a mature adipocytes. Specifically, the present invention relates to the surprising discovery that autophagy is critical for the cellular remodeling required during pre-adipocyte differentiation into mature adipocyte. By targeting and inhibiting one or more mechanisms in autophagy, adipocyte maturation is also inhibited, thus, providing a novel a pathway to prevent, mitigate and/or treat weight gain, obesity and associated diseases, such as type II diabetes.

### CROSS REFERENCE TO RELATED APPLICATIONS

The instant application claims 35 U.S.C. §119(e) priority to U.S. Provisional Patent Application Ser. No. 61/189,628 filed Aug. 20, 2008, the disclosure of which is incorporated by reference herein.

### STATEMENT REGARDING FEDERALLY SPONSORED RESEARCH OR DEVELOPMENT

The invention described herein was supported in whole or in part by grants from The National Institute of Health (Grant Nos. 1R01 CA116088-01A1, 1R01 AG030081-01A1, and 5F31 GM078857-02) and the Department of Defense (Grant No. DOD BC060538). The government has certain rights in the invention.

### FIELD OF THE INVENTION

The present invention relates to methods of preventing, mitigating, or treating weight gain or obesity in patients by administering one or more autophagy inhibitors, thereby, preventing the differentiation process of pre-adipocyte cells into mature adipocytes.

### BACKGROUND OF THE INVENTION

Obesity is becoming a pandemic in the United States and most other developed countries, where it represents a real threat to the health of people and a huge burden to the health care systems. Obesity is a direct outcome of accumulation, maturation, and expansion of adipocytes (fat cells). In humans and other mammals there are two types of adipose tissue, white adipose tissue (WAT) and brown adipose tissue (BAT). WAT is the predominant type and are highly differentiated cells with a very distinctive structure in which nearly the entire cell volume is occupied by one large lipid droplet while other cellular components, including the nucleus and cytoplasm, localize peripherally and occupy minimal space. The lipid droplet in these mature adipocytes can serve as a depot for the excess fatty acids in the body. These adipocytes are derived from pre-adipocytes that are morphologically similar to fibroblast cells and structurally distinctive from the mature adipocytes. The differentiation of a mature adipocyte from a fibroblast-like pre-adipocyte requires highly coordinated and massive cellular remodeling processes.

Autophagy is a membrane trafficking process leading to lysosomal degradation. It is one of the two major cellular degradation processes (the other one is the ubiquitin dependent proteolysis). Autophagy is initiated by the emergence of a double membrane structure in the cytoplasm, which expands to engulf and sequester a portion of the cytoplasm, creating the hallmark double-membrane vesicle called autophagosome or autophagic vacuole. Fully-matured autophagosomes translocate towards and fuse with lysosomes. The cargo of the autophagosomes is then released to the lysosome where it is degraded. The molecular machinery of autophagy has been recently identified. The genes encoding the essential components of the machinery are named atg (autophagy-related) genes, which are highly conserved from yeast to mammals. Deletion of one of the essential atg genes totally inhibits the autophagy process. In addition, a number of pharmacological inhibitors of autophagy are available.

Previously, autophagy modulators have been primarily explored for the treatment of cancer. U.S. patent application Ser. No. 11/814,365, in particular, relates to methods of treating patients having a glycolysis dependent cancer by co-adminstering a compound to inhibit glycolysis along with an autophagy inhibitor. Along these lines, multiple autophagy inhibitors were identified.

Using autophagy for an opposing purpose, U.S. patent application Ser. No. 11/119,569 teaches the treatment of cancer by stimulating autophagy to induce apoptosis. Specifically, a key enzyme in the autophagy pathway, ATG7, is administered to the patient, thereby, stimulating autophagy in the carcinoma and inducing cell death.

PCT International Application No. PCT/US08/059129 further provides a family of autophagy modulating, both inhibiting and stimulating, compounds. These compounds are taught not only for use with cancer, but also inflammatory diseases, autoimmune diseases, cardiovascular diseases (e.g., reperfusion injury, ischemic cardiac disease), infectious diseases (e.g., viral infections, bacterial infections), neurodegenerative diseases (e.g., Huntington's disease, Alzheimer's disease), and protein folding disorders (e.g., Alzheimer's disease, cystic fibrosis).

Prior to the instant application, however, there was no link between obesity and the autophagy pathway. Indeed, prior to the instant application, it was unknown what role autophagy played, if any, in the differentiation process of pre-adipocytes into mature adipocytes. The instant invention provides such a newly discovered link and illustrates potential uses of this link for treating and/or preventing obesity, as well as identifying one or more potentially new compounds that inhibit adipocyte cell (i.e. WAT) formation.

### SUMMARY OF THE INVENTION

The present invention relates to methods of preventing, mitigating or treating weight gain or obesity in patients by administering one or more autophagy inhibitors, thereby, preventing the differentiation process of pre-adipocyte cells into a mature adipocytes (i.e. WAT). Specifically, the present invention relates to the surprising discovery that macroautophagy (or autophagy in short) is critical for the cellular remodeling required during pre-adipocyte differentiation into a mature adipocyte. By targeting and inhibiting one or more of the mechanisms of autophagy, adipocyte maturation is also inhibited, thus, providing a novel a pathway to prevent or mitigate weight gain, obesity and obesity related diseases, such as type II diabetes. Additionally, because adipocytes turn-over at a rate of approximately 10% annually (citation below), the instant invention is similarly advantageous for treating weight gain and obesity.

Based on the foregoing, the instant invention relates to the administration of one or more autophagy inhibitors for the purpose of inactivating autophagy genetically and pharmacologically to inhibit adipocyte maturation and specifically promote death of the differentiating cells. This invention can be used to effectively and specifically prevent, mitigate, and/or treat pathological conditions related to the accumulation of excess mature adipocytes such as weight gain, obesity, as well as the diseases related to obesity. In one embodiment, the autophagy inhibitors of the instant invention may target autophagosome-lysosome fusion. Alternatively, the autophagy inhibitor may inhibit the expression of an atg gene, such as but not limited to atg1, atg5, atg6, or atg7. As a further alternative, the autophagy inhibitor may inhibit the activity of one or more ATG proteins, such as but not limited to ATG1, ATG5, ATG6, and ATG7.

The autophagy inhibitor may be compound. Such a compound may include hydroxychloroquine, and analogs thereof, or similar compounds identified herein or otherwise discussed in the art. The autophagy inhibitor may also include other molecular or biologic agents, such as but not limited to a nucleic acid inhibitor. Such nucleic acids may include, but are not limited to an encoding DNA enzyme, an antisense RNA, an siRNA, a shRNA, or aptamer, and can be designed based on criteria well known in the art or otherwise discussed herein.

Numerous clinical therapeutic indications envisioned for administration of an effective amount of one or more of the autophagy inhibitors herein include, but are not limited to, any preventative, mitigating and/or treating regiment targeting, generally, the pathological conditions relating to weight gain or obesity. In one embodiment, for example, administration of the autophagy inhibitors targets the development of a weight gain or obesity condition as a side effect of taking certain prescription drugs, such as, but not limited to, Lithium, Valproate, Depakote, Zyprexa, Paxil, Ergenyl, Absenor, Orfilir, Chlorpromzine, Elavil, Tofranil, Xeroxat, Cipramil, Sertralin, Zoloft, Cortisone, Prednisone, Follimin, Follinett, Neovletta, Sandomigrin, Ergenyl, Trypizol.

Autophagy inhibitors may be administered to prevent/mitigate/treat the development of weight gain or an obesity condition as a result of known medical conditions or other genetic factors. Such conditions include, but are not limited to, hypothyroidism, Cushing's syndrome, growth hormone deficiency, Prader-Willi syndrome, Bardet-Biedl syndrome, MOMO syndrome. Examples of these genetic factors include but not limited to: polymorphism of certain genes, such as the leptin receptor and melanocortin receptor belonging to certain ethnic groups.

Autophagy inhibitors may be administered to prevent/mitigate/treat the development of weight gain or an obesity condition related to smoking cessation or to prevent/mitigate/treat the development of weight gain or an obesity condition associated with sedentary lifestyle or dietary factors.

Autophagy inhibitors may be administered for preventing, mitigating, and treating pathological conditions attributed to or in conjunction with weight gain or obesity. One such condition is type II diabetes. Specifically, the data discussed below illustrates that administration of one or more autophagy inhibitors lead to alteration of adipose tissues in such a way that the subject has an increased sensitivity to insulin. To this end, the effect of administration is to counteract the insulin deficiency observed with type II diabetes. The instant invention, however, is not limited to treating type II diabetes and may treat other conditions including, but not limited to, the following: (1) cardiovascular diseases; (2) Hyperlipidimia; (3) Certain cancers; (4) Gallbladder disease and gallstones; (5) Osteoarthritis; (6) Gout; and (7) Breathing problems, such as sleep apnea and asthma.

One or more autophagy inhibitor of the present invention, either alone or in combination with another active ingredient, may be synthesized and administered as a therapeutic composition using dosage forms and routes of administration contemplated herein or otherwise known in the art. Dosaging and duration will further depend upon the factors provided herein and those ordinarily considered by one of skill in the art. To this end, determination of a therapeutically effective amount are well within the capabilities of those skilled in the art, especially in light of the detailed disclosure and examples provided herein.

### BRIEF DESCRIPTION OF THE FIGURES

FIG. 1 illustrates a schematic of the role of each of the four groups of atg proteins that form the core autophagy machinery.

FIG. 2 illustrates a schematic of the first and second conjungation systems that form the core autophagy machinery.

FIG. 3 illustrates that autophagy was activated in wild type MEFs during adipogenesis (A) Primary atg5+/+ MEFs were induced for adipogenesis. At indicated time points, the progress of differentiation was analyzed. Cells were observed under microscope (Olympus IX70) equipped with relief contrast objectives (10× and 40×, for low and high magnification, respectively.) Selected regions in pictures of low magnification (within the squares) are shown below with high magnification. (B). Electron microscopy analysis of primary atg5+/+ MEFs 0, 2, or 6 days after differentiation induction, as indicated. Upper panel shows micrographs of low magnification, and the lower panel shows high magnification of the selected regions (square) in the upper panel. Arrows indicate autophagosomes. (C). Ratio of the volume of autophagosomes to cytosol. The volume of autophagosomes and cytosol were determined by point counting of 15-20 micrographs of cells 0, 2, or 6 days after differentiation induction, as indicated. ***P<0.001. Student ttest. (D) and (E). Immunoblotting assays of differentiating cells. The cells at indicated time points with (D) or without (E) differentiation induction were harvested and immunoblotting assays were performed with LC3, Atg12, p62, or RAN antibodies, as indicated. The levels of RAN served as a loading control. The data are representative results from three independent experiments.

FIG. 4 illustrates that autophagy deficient primary atg5−/− MEFs exhibited reduced efficiency in adipogenesis. Primary atg5+/+ or atg5−/− MEFs were induced for adipogenesis. At indicated time points, the progress of differentiation was analyzed. (A). Cells were observed under microscope (Olympus IX70) equipped with relief contrast objectives (10× and 40×, for low and high magnification, respectively.) Selected regions in pictures of low magnification (within the squares) are shown below with high magnification. (B) Cells were stained with the lipid dye Bodipy 493/503 and observed with microscope under phase contrast objectives (20×). (C). 14 days post-differentiation inductions, cells were stained with the lipid dye Oil Red-O and hematoxylin, and observed under phase contrast microscope. (D). Cells grown on cover slip were stained with Oil Red-O at indicated time points and Oil Red-O was extracted and measured by spectrometry. These data represent results from experiments with cells derived from four independent pairs of embryos of three independent breeding parents.

FIG. 5 illustrates that quantitative PCR analysis of the expression of a subset of adipogenesis marker genes. mRNA were extracted from the atg5+/+ and atg5−/− cells at Day 0 or Day 6 of differentiation and analyzed by quantitative PCR. The graphic representations show relative expression levels of each adipogenesis related gene, as indicated, as compared to the normalizer gene Wbp11NORM. * denote values that were undetectable. Error bars represent one standard deviation.

FIG. 6 illustrates that time-lapse microscopy analysis of adipogenesis in the atg5+/+ and atg5−/− MEFs. Primary MEFs were treated to induce adipocyte differentiation. Three days after induction, time-lapse microscopy with relief contrast lens was performed to monitor the progression of differentiation. Panels (A), (B), (C), and (D) are picture frames taken from two-day movie clips showing the continuous morphologic changes during differentiation. Areas in square regions of Panels A and C are enlarged below to show detail in Panels B and D, respectively. White arrows in (B) point to a growing lipid droplet; and black arrows in (C), and (D) point to cells undergoing abortive differentiation. The data represent results from experiments performed with three independent pairs of MEFs.

FIG. 7 illustrates that differentiating atg5−/− MEFs exhibited higher rates of apoptosis. (A). Primary atg5+/+ or atg5−/− MEFs were induced for adipogenesis. The progress of differentiation and apoptotic cell death was analyzed with Bodipy 493/503 staining (green), DAPI staining (blue) and TUNEL assay (red), respectively. The pictures showed cells at Day 6 post-differentiation induction. Representative low (with a scale bar of 50 μm) and higher (with a scale bar of 10 μm) magnification pictures are shown. (B). Quantification of the TUNEL positive cells as a percentage of Bodipy 493/503 positive cells at the indicated time points. Total number of Bodipy 493/503 positive cells and total number of both TUNEL positive cells and Bodipy 493/503 positive cells in randomly selected regions were counted and the percentage was calculated. The data are representative results from three independent experiments. ** P<0.01; Student t-test.

FIG. 8 illustrates that the atg5−/− mice had less subcutaneous fat cells. atg5−/− embryos (E18.5) and neonatal pups (within 12 hours after birth) and their wild type littermates were obtained and the transverse sections at the level of scapulae were analyzed by immunofluorescence microscopy with primary antibody against perilipin A and FITC conjugated secondary antibody. (A). Subcutaneous regions of embryos showing perilipin A positive adipocytes. (B). Subcutaneous regions of neonatal pups showing perilipin A positive adipocytes. (C). quantification of (A). Total number of perilipin A positive cells in subcutaneous regions of three adjacent scapulae sections were counted and averaged. (D). quantification of (B). Total number of perilipin A positive cells in subcutaneous regions of three adjacent scapulae sections were counted and averaged. The data are representative results from three independent pairs of pups born to two independent pairs of parents and three pairs of embryos born to three pairs of parents. ** P<0.01; *** P<0.001. Student t-test.

FIG. 9 illustrates that chloroquine significantly reduced the efficiency of adipogenesis in primary MEFs. Wild type primary MEFs were induced for adipogenesis with or without co-treatment of 10 μM chloroquine (CQ). Differentiation progress was then monitored by: (A). microscopy analysis; (B). lipid analysis with Bodipy 493/503 staining (14 days after differentiation induction); (C). lipid analysis by spectrometry of Oil Red-O staining (14 days after differentiation induction). (D) and (E) are controls that show that chloroquine was non-toxic (D) and efficacious in inhibiting autophagosome fusion with lysosome and in inhibiting autophagy flux (E) at the experimental concentration. (D). Tunel assays of wild type MEFs treated with 10 μM chloroquine for 4 days compared with cells without chloroquine treatment. Cells treated with 10 μM staurosporine (STS) for 6 hr was used as a positive control. (E). Cells treated with/or without 10 μM chloroquine at different time points were harvested, immunoblotting assays were performed with LC3, p62, or RAN antibodies, as indicated. The levels of RAN served as a loading control. The results represent three independent experiments. *P<0.05; Student t-test.

FIG. 10 illustrates that adipose-specific atg7 knockout mice exhibited reduced body weight and white adipose tissue mass. (A). Immunoblotting analyses of white adipose tissues (female, uterine WAT) from control (atg flox/flox) and adipose-specific atg7 conditional knockout (atg7flox/flox; ap2-Cre) mice using indicated antibodies (Atg12-Atg5 conjugate was detected with an anti-Atg12 antibody). (B). Upper panel, body weight chart of control (female, n=12) and atg7 conditional knockout (female, n=11) mice from 4 to 18 weeks (***P<0.001, Student's ttest). Lower panel, a two-week food intake chart of control (female, n=6) and atg7conditional knockout (female, n=6) mice starting from week 11. (C). Representative pictures of control and atg7 conditional knockout mice at the age of 20 weeks, showing gonadal (upper panel) and interscapular (lower panel) white adipose tissues (WAT) as indicated by arrows. (D). Representative pictures (upper panel) of gonadal fat pad (uterine fat in female and epididymis fat in male) and quantification (lower panel) from control (male, n=10; female, n=12) and atg7 conditional knockout (male, n=5; female, n=6) mice at the age of 18˜20 weeks. ***P<0.001, Student's t-test.

FIG. 11 illustrates that histological and immunofluorescence analysis of gonadal WAT from control and atg7 conditional knockout mice. (A-F). Representative microscopic pictures of H&E stained sections of uterine WAT from control (atg7flox/flox, A and D) and adipose-specific atg7 conditional knockout mice (atg7flox/flox; aP2-Cre, B-C and E-F). Selected regions in pictures A-C (within the squares) were shown below with high magnification (D˜F). (G˜L). Representative microscopic pictures of immunofluorescence assays of uterine WAT from control (G and J) and atg7 conditional knockout mice (H-I and K-L) with Perilipin A antibody. G-I were pictures of low magnification and J-L were pictures of high magnification. (M˜O). Quantification of average cell volume, lipid droplet volume, and percentage of multilocular cells, as indicated, of uterine WAT from control and atg7 conditional knockout mice. Detail methods for quantification were described in Material and Methods. ***P<0.001, Student's t-test. The data showed representative results of tissues from six pairs of female mice (control and atg7 knockout).

FIG. 12 illustrates that adipose-specific atg7 knockout mice accumulated more mitochondria in gonadal WAT. (A). Immunofluorescence analyses of gonadal WAT (uterine WAT) from control (atg7flox/flox) and adipose-specific atg7 conditional knockout mice (atg7flox/flox; aP2-Cre) with COX II antibody, observed under microscope with low (upper panel) and high magnification (lower panel). The nuclei were stained with DAPI. The data were representative results from three pairs of mice. (B). Electron microscopic pictures of adipocytes from uterine WAT of control and adipose-specific atg7 knockout mice. Selected regions in pictures of low magnification (within the squares) were shown below with high magnification. LD, lipid droplet; N, nucleus; arrows indicate mitochondria in the control tissues.

FIG. 13 illustrates that immunofluorescence and electron microscopic analysis of brown adipose tissues. (A). Immunofluorescence analyses of interscapular brown adipose tissues (iBAT) from control (atg7flox/flox) and adipose-specific atg7 conditional knockout mice (atg7flox/flox; aP2-Cre) at the age of 19 weeks with Perilipin A antibody, observed under microscope with low (upper panel) and high magnification (lower panel). Nuclei were stained with DAPI. (B). Quantification of the volume of the largest lipid droplets of iBAT from control and atg7 conditional knockout mice. 50 largest lipid droplets were selected from each perilipin A immunofluorescence picture and the size was measured and the volume calculated. The data showed representative results of tissues from six pairs of female mice (control and atg7 knockout). ***P<0.001, Student's-test. (C). Representative electron microscopic pictures of iBAT from control and atg7 conditional knockout mice. Selected regions in pictures of low magnification (within the squares) were shown below with high magnification.

FIG. 14 illustrates that autophagy deficient primary atg7−/− MEFs exhibited reduced efficiency in adipogenesis. Primary atg7+/+ or atg7−/− MEFs were induced for adipogenesis. At indicated time points, the progress of differentiation was observed and analyzed. (A). Cells were observed under a microscope equipped with relief contrast (10×) and phase contrast (10×) objectives. (B). Cells were stained with the lipid dye Bodipy 493/503 and observed with microscopy under phase contrast objectives (20×). (C). 14 days after differentiation inductions, cells grown on cover slips were stained with the lipid dye Oil Red-O and scanned. (D). Cells grown on cover slips were stained with Oil Red-O at indicated time points and Oil Red-O was extracted and measured by spectrometry. These data represent results from experiments with cells derived from two pairs of embryos.

FIG. 15 illustrates that analyses of metabolic parameters of the adipose-specific atg7 conditional knockout mice. Fasting plasma levels of triglyceride (A), total cholesterols (B), glycerol (C), and free fatty acids (D) in control (atg7flox/flox, male, n=6) and adipose-specific atg7 conditional knockout (atg7flox/flox;aP2-Cre, male, n=6) mice. (E). Insulin tolerance tests. Control (male, n=6) and atg7 conditional knockout (male, n=6) mice were fasted for 5 hours before receiving an intraperitoneal injection of 0.75 Unit/kg insulin and blood samples were taken at indicated time points. (F). Glucose tolerance tests. Control (male, n=6) and atg7 conditional knockout (male, n=6) mice were fasted overnight before receiving an intraperitoneal injection of 2 g/kg glucose and blood samples were taken at indicated time points. *P<0.05; **P<0.01; ***P<0.001, Student's t-test. The data were representative results from two independent experiments.

FIG. 16 illustrates that comparison of weight gain under high-fat diet between the control and adiposespecific atg7 conditional knockout mice. (A). Body weight chart of control (atg7flox/flox, male, n=9) and adipose-specific atg7 conditional knockout mice (atg7flox/flox;aP2-cre, male, n=6) fed with normal diet (ND) or high fat diet (HFD) from the age of 8 to 16 weeks (*P<0.05, Student's t-test). (B). One-week HFD food intake chart of control (male, n=6) and atg7 conditional knockout (male, n=6) mice starting from week 14.

### DETAILED DESCRIPTION OF THE INVENTION

Definitions

As used herein, the term “autophagy inhibitor” refers to a compound or any biological agent that decreases the level of autophagy in a cell undergoing autophagy in its presence, compared to the level of autophagy in a cell undergoing autophagy in its absence.

As used herein, the term “biological agent” or “biological agents” include any agent known in the art such as, but not limited to, proteins or protein-based molecule, such as a mutant ligand, antibody, or the like, and nucleic acids or nucleic acid-based entities and the vectors used for their delivery.

As used herein, the term “compound” or “compounds” refers to conventional chemical compounds (e.g., small organic or inorganic molecules). To this end, the terms small molecule and compounds are interchangeable.

As used herein, with respect to administering an autophagy inhibitor, the terms “mitigate” or “mitigating” refers to reducing the progression of a disease or condition. It may include executing a protocol, which may include administering one or more drugs to a patient (human or otherwise), in an effort to reduce signs or symptoms of the disease.

As used herein, the term “obese” or “obesity” refers to a condition in which there is an excess of body fat in a subject. Obesity may be due to any cause, whether genetic or environmental. The operational definition of obesity is based on the Body Mass Index (BMI), which is calculated as body weight per height in meters squared (kg/m²). “Obesity” also refers to a condition whereby an otherwise healthy subject has a Body Mass Index (BMI) greater than or equal to 30.0 kg/m², or a condition whereby a subject with at least one co-morbidity has a BMI greater than or equal to 27.0 kg/m². An obese subject is a subject with a Body Mass Index (BMI) greater than or equal to 30.0 kg/m² or a subject with at least one co-morbidity with a BMI greater than or equal to 27.0 kg/m². An obese subject may have a BMI of at least about any of 31.0, 32.0, 33.0, 34.0, 35.0, 36.0, 37.0, 38.0, 39.0, and 40.0. An overweight subject is a subject with a BMI of 25.0 to 29.9 kg/m².

As used herein, with respect to administering an autophagy inhibitor, the terms “prevent,” or “preventing” refers to prophylactic treatment for halting a disease or condition. It may include executing a protocol, which may include administering one or more drugs to a patient (human or otherwise), in an effort to prevent signs or symptoms of the disease. In certain embodiments, prophylactic treatment prevents worsening of a disease or condition.

As used herein, the terms “siRNA molecule,” “shRNA molecule,” “RNA molecule,” “DNA molecule,” “cDNA molecule” and “nucleic acid molecule” are each intended to cover a single molecule, a plurality of molecules of a single species, and a plurality of molecules of different species.

As used herein, the term “siNA” is intended to cover siRNA as well as siDNA sequences. The term “shNA” is intended to cover shRNA as well as shDNA sequences.

As used herein, the term “subject” refers to any animal (e.g., a mammal), including, but not limited to humans, non-human primates, rodents, and any other animal, which is to be the recipient of a particular treatment. Typically, the terms “subject” and “patient” are used interchangeably herein in reference to a human subject.

As used herein, with respect to administering an autophagy inhibitor, the terms “treat,” “treating,” or “treatment” refers to therapeutic treatment for halting or reducing a disease or condition. It may include executing a protocol, which may include administering one or more drugs to a patient (human or otherwise), in an effort to alleviate signs or symptoms of the disease. In certain embodiments, therapeutic treatment prevents worsening of a disease or condition.

The present invention relates to methods of preventing, mitigating, or treating weight gain or obesity in patients by administering one or more autophagy inhibitors, thereby, preventing the differentiation process of pre-adipocyte cells into a mature adipocyte. Specifically, the present invention relates to the surprising discovery that macroautophagy (or autophagy in short) is critical for the cellular remodeling required during pre-adipocyte differentiation into mature adipocyte. To this end, the invention illustrates that targeting one or more of the mechanisms used in autophagy greatly inhibits adipocyte maturation and promotes adipocyte death, thus, providing a novel a pathway to prevent, mitigate and/or treat weight gain, obesity and obesity related diseases. Additionally, because adipocytes turn-over at a rate of approximately 10% annually, the instant invention is similarly advantageous to treat existing obesity and obesity related diseases.

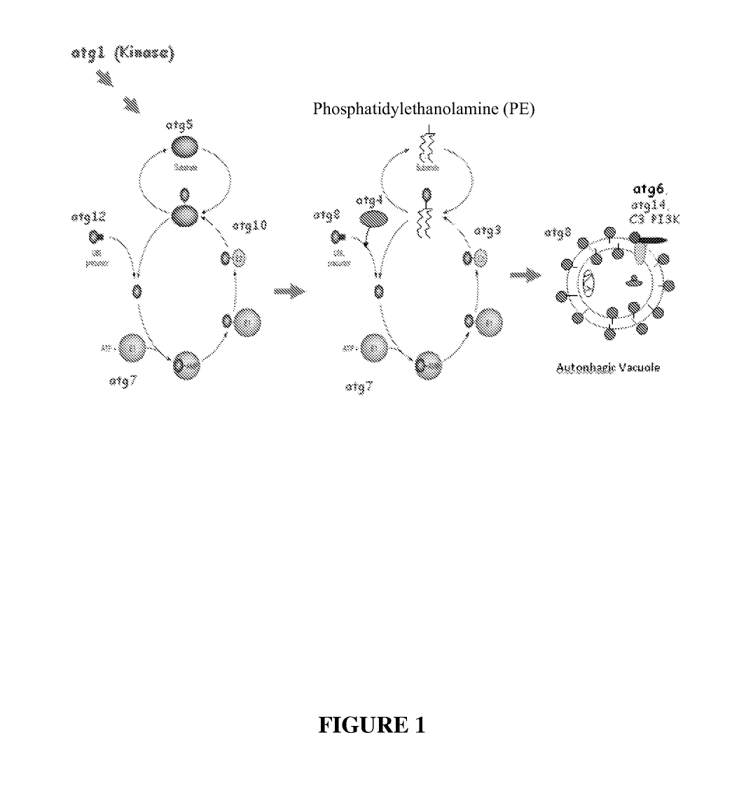

Referring to FIG. 1, an illustration of the process in autophagy is provided, including several of the atg proteins that form the core autophagy machinery. These proteins can be functionally divided into four groups. The first group is comprised of atg1, which is a kinase playing a regulatory role in autophagy activation. The second group is comprised of atg5, atg12, atg7 and atg10 and constitute a ubiquitin-like first protein conjugation system. Atg12 is a ubiquitin like protein, and atg7 and atg10 are a El and E2 like enzymes, respectively. Through the enzymatic activities of atg7 and atg10, the small ubiquitin like protein atg12 is conjugated to atg5. The third group is comprised of atg8, agt7, and atg3, which constitute a second unique ubiquitin-like protein conjugation system. Atg8 is a ubiquitin like protein, and atg7 and atg3 are a E1 and E2 like enzymes, respectively. Through the function of atg7 and atg3, the ubiquitin like protein atg8 is conjugated to phospholipid, a very unique feature. The fourth group of proteins include atg6, atg14 and a type III PI3 kinase. These proteins form a complex that locate on the autophagosome are involved in targeting or recruitment of a lysosome.

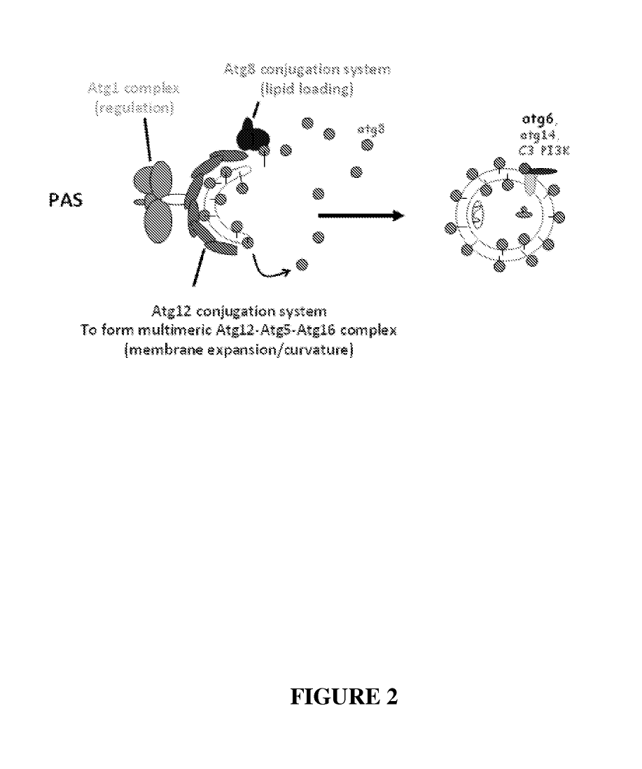

Referring to FIG. 2, the first protein conjugation system allows atg12 conjugate to atg5, which triggers a process to form a multimeric complex of the atg12-atg5 repeating units. This complex forms a scaffold for the expansion of the autophagosome. The second protein conjugation system allows atg8 to conjugate to the phospolipids. It is believed that this complex is involved in loading soluble phospholipid to the autophagosome, with the atg6 complex likely playing a role in lysosome targeting.

Using the foregoing, the examples below illustrate these mechanistic requirements of autophagy are required for pre-adipoctye cell differentiation into mature adipocyte. As illustrated below, wild type primary mouse embryonic fibroblasts (MEF) undergo massive adipocyte differentiation when induced using standard adipocyte differentiation protocols. MEF cell types derived from autophagy deficient mice (e.g. atg5−/− or atg7−/− cells), however, demonstrated significantly reduced adipocyte maturation and promotion of adipocyte death. In other words, the absence of one or more of the required mechanisms (e.g. proteins, mRNA, etc) for autophagy surprisingly inhibits the maturation of pre-adipocyte tissue when induced under standard protocols.

This effect was further confirmed by the administration of hydroxylchloroquine, a known late inhibitor of autophagosome-lysosome fusion used for treating malaria, rheumatoid arthritis and lupus. Wild type primary mouse adipocytes were treated to induce adipocyte differentiation under the same standard differentiation protocol, in the absence or presence of various concentrations of hydroxychloroquine. Consistent with the results above, the cells underwent normal adipocyte differentiation in the absence of hydroxychloroquine, and, in the presence of 10 micro molar hydroxychloroquine, the primary MEFs stalled at the initial stage of adipocyte differentiation. Those cells that did differentiate eventually died, however, and the viability of the undifferentiated cells was not apparently affected by hydroxychloroquine treatment. Lower concentrations of hydroxychloroquine (1 micro molar and 5 micro molar) had inhibitory effect on blocking adipocyte differentiation, albeit to a lesser degree compared to 10 micro molar concentration. It is noteworthy that these concentrations are within the steady-state blood concentration ranges of hydroxychloroquine during treatment of chronic diseases such as rheumatoid arthritis.

Based on the foregoing, the instant invention relates to the administration of one or more autophagy inhibitors for the purpose of inactivating autophagy genetically and pharmacologically to inhibit adipocyte maturation and specifically promote death of the differentiating cells. This invention can be used to effectively and specifically mitigate or prevent pathological conditions related to adipocyte maturation such as weight gain, obesity, and associated diseases.

The instant invention may also be used to effectively treat pathological conditions related to the accumulation of excess adipoctes. Specifically, in adult humans the number of adipocytes remains relatively stagnant with approximately 10% of the cell being renewed and regenerated (i.e. turned-over) annually. By administering one or more autophagy inhibitors for the purpose of inhibiting adipocyte maturation, one would effectively reduce the adipocyte mass by at least 10% each year, thereby, treating obesity. For a review of adipocyte turn-over see Spalding, Kirsty L. et al. Dynamics of fat cell turnover in humans. Nature. June 2008 453:783-787, the contents of which are incorporated by reference herein.

In one embodiment, the autophagy inhibitor may be a compound that targets one or more mechanisms within the autophagy pathway (e.g. enzymes, proteins, mRNA expression, etc). In one embodiment, the compound is comprised of hydroxychloroquine. However, the instant invention is not so limited and other autophagy inhibitory compounds are also applicable as autophagy inhibitors for the prevention, mitigation and/or treatment of weight gain or obesity. Such compounds may include, but are not limited to 3-methyladenine, 5-amino-4-imidazole carboxamide riboside (AICAR), okadaic acid, N6-mercaptopurine riboside, autophagy-suppressive algal toxins which inhibit protein phosphatases of type 2A or type 1 analogues of cAMP, adenosine, wortmannin, cefamandole, monensin, astemizole, spiramycin, (1S,9R)-beta-hydrastine, carnitine, tomatine, K252A, atranorin, tetrandrine, amlodipine, benzyl isothiocyanate, pristimerin, homochlorcyclizine (e.g., homochlorcyclizine dihydrochloride), fluoxetine (e.g., fluoxetine hydrochloride), bafilomycin A1, wiskostatin, monensin, quinacrine, nocodazole, vinblastine, colchicine, puromycin, bepridil, spiramycin, migericin, 2-methylcinngel, amiprilose, carnitine, tyrphostin 9, salinomycin, PP1, lavendustin A, ZL3VS, astemizole, G06976, RWJ-60475-(AM)3, D609, mefenamic acid, cytochalasin D, E6 berbamine, beta-peltatin, aesculin, GF-109203D, benzyl isothiocyanate, monensin, podophyllotoxin, thimerosal, maprotiline hydrochloride, vinblastine, norethindrone, gramacidin, sunitinib, UCNO1, PKC412, and ruboxistaurin. Compounds may also include those having a bis-indolyl maleimide core such as K252A, Go6976, and GF-109203X, as well as analogs thereof, as set forth in PCT Published International Application No. PCT/US08/059129, the contents of which are incorporated by reference herein. The compounds may further include those within U.S. patent application Ser. No. 11/814,365, the contents of which are incorporated by reference herein. One of ordinary skill in the art would appreciate that chemical analogs of one or more of the foregoing compounds would achieve similar results.

In addition to the use of compounds described above, autophagy inhibitors may also include other molecular or biologic agents. In one embodiment, the autophagy inhibitor is a nucleic acid molecule capable of inhibiting the expression of one or more proteins within the autophagy pathway. Such nucleic acids may include, but are not limited to an encoding DNA enzyme, an antisense RNA, an siRNA, a shRNA, dsRNA or aptamer, and can be designed based on criteria well known in the art or otherwise discussed herein. As noted above, the autophagy machinery has been identified and most of the genes/proteins required for autophagy activation are well established. Expression products for one or more atg genes, as well as their interacting proteins, may be targeted, wherein deletion or inhibition of one of these essential genes such as atg1, atg5, atg6 (beclin1), or atg7 effectively diminishes or severely reduces autophagy activity. To this end, the nucleic acid molecule may be specifically targeted to the expression products of one or more of these genes.

DNA enzymes may be comprised of magnesium-dependent catalytic nucleic acids of DNA that can selectively bind to an RNA substrate, such as an atg RNA substrate, by Watson-Crick base-pairing and potentially cleave a phosphodiester bond of the backbone of the RNA substrate at any purine-pyrimidine junction. As understood in the art, DNA enzymes are comprised of two distinct functional domains: a 15-nucleotide catalytic core that carries out phosphodiester bond cleavage, and two hybridization arms flanking the catalytic core; the sequence identity of the arms can be tailored to achieve complementary base-pairing with target RNA substrates. In the instant invention, a DNA enzyme may be used that has complementary regions that can anneal with regions on the transcript of an Atg gene such that the catalytic core of the DNA enzyme is able to cleave the transcript and prevent translation.

An antisense RNA molecule would similarly contain a sequence that is complementary to the RNA transcript of an Atg gene, and which can bind to the Atg transcript, thereby reducing or preventing the expression of the Atg gene in vivo. The antisense RNA molecule will have a sufficient degree of complementarity to the target mRNA to avoid non-specific binding of the antisense molecule to non-target sequences under conditions in which specific binding is desired, such as under physiological conditions.

Nucleic acid molecules that silence genes using RNA interference (RNAi) may also be used. siRNAs (short interfering RNAs) are double-stranded RNA (dsRNA) molecules that induce the sequence-specific silencing of genes by the process of RNA interference (RNAi) in multiple organisms, including humans. An siRNA typically targets a 19-23 base nucleotide sequence in a target mRNA. Naturally occurring siRNAs tend to be 21-28 nucleotides in length and occur naturally in cells. However, synthetic siRNAs have been used to specifically target gene silencing in mammalian cells. Alternative aspects of siRNA technology include chemical modifications that increase the stability and specificity of the siRNAs, and a variety of delivery methods and in vivo model systems. siRNA sequences can for example be designed using software algorithms that are commercially available. For example, the algorithm BLOCK-iT™ RNAi Designer (Invitrogen, California), can be used to select appropriate sequences for an siRNA directed against an Atg gene such as atg1 atg5, atg6 or atg7. Such siRNA may be any one or more of the sequences, or homologues thereof, set forth in PCT International Application No. PCT/CA06/001822, the contents of which are incoporated by reference herein.

Small hairpin RNA (shRNA) are also contemplated for RNAi of autophagy expression. shRNA is a sequence of RNA that makes a tight hairpin turn that can be used to silence gene expression via RNA interference. These hairpin structures, once processed by the cell, are equivalent to siRNA molecules and are used by the cell to mediate RNAi of the desired protein. The use of shRNA has an advantage over siRNA transfection as the former can lead to stable, long-term inhibition of protein expression. Such shRNA may be designed using standard methodologies known in the art and may include, but is not limited to, the methodologies and shRNA, or homologues thereof, as set forth in U.S. application Ser. No. 11/814,365, the contents of which are incorporated by reference herein.

The autophagy inhibiting nucleic acids of the instant invention can be introduced into cells in vitro or ex vivo using techniques well-known in the art, including electroporation, calcium phosphate co-precipitation, microinjection, lipofection, polyfection, and conjugation to cell penetrating peptides (CPPs). In one embodiment, such nucleic acid can be introduced into cells in vivo by endogenous production from an expression vector(s) encoding the appropriate sequences. Such expression vectors may be comprised of any expression vectors known in the art that is operably linked to a genetic control element capable of directing expression of the nucleic acid within a cell. Expression vectors can be transfected into cells using methods generally known to the skilled artisan.

Biological agents as autophagy inhibitors are not necessarily limited to nucleic acids and may be comprised of any other agents otherwise known in the art that may be contemplated for inhibiting the expression of a gene required for autophagy or inhibiting the action of an enzyme required for autophagy. Such agent may include, but are not limited to antibodies, ribozymes, proteins, or other biological agents known in the art for such purposes.

As provided herein, the clinical therapeutic indications envisioned for administration of an effective amount of one or more of the autophagy inhibitors herein include, but are not limited to, any preventative, mitigating and/or treatment regiment targeting, generally, the pathological conditions relating to weight gain or obesity. In one embodiment, administration of the autophagy inhibitors targets the development of weight gain or an obesity condition as a side effect of taking certain prescription drugs. Such drugs include but are not limited to: Lithium (for manic bipolar disorders), anti-seizure medicine (e.g. Valproate, Depakote); antipsychotics (e.g. Zyprexa, Paxil, Ergenyl, Absenor, Orfilir, Chlorpromzine); mood stabilizers (e.g. Elavil, Tofranil, Xeroxat, Cipramil, Sertralin, Zoloft); steroids (e.g. Cortisone, Prednisone); oestrogen (e.g. Follimin, Follinett, Neovletta); migraine medicines (e.g. Sandomigrin, Ergenyl, Trypizol). Co-treatment of patients taking one or more of these or similar medication with one or more autophagy inhibitors of the instant invention would greatly reduce the adverse effect.

In an alternative embodiment, autophagy inhibitors may be administered to prevent/mitigate/treat the development of weight gain or an obesity condition as a result of known medical conditions. Such conditions include, but are not limited to, hypothyroidism, Cushing's syndrome, growth hormone deficiency, Prader-Willi syndrome, Bardet-Biedl syndrome, MOMO syndrome. Autophagy inhibitors also may be administered to prevent/mitigate/treat the development of weight gain or an obesity condition as a result of genetic pre-disposition. The examples of these genetic factors include but not limited to: polymorphism of certain genes, such as leptin receptor and melanocortin receptor belonging to certain ethnic groups.

In further embodiments, autophagy inhibitors may be administered to prevent/mitigate/treat the development of weight gain or an obesity condition associated with smoking cessation. Along these lines, autophagy inhibitors may be administered to prevent/mitigate/treat the development of weight gain or an obesity condition associated with sedentary lifestyle or dietary factors.

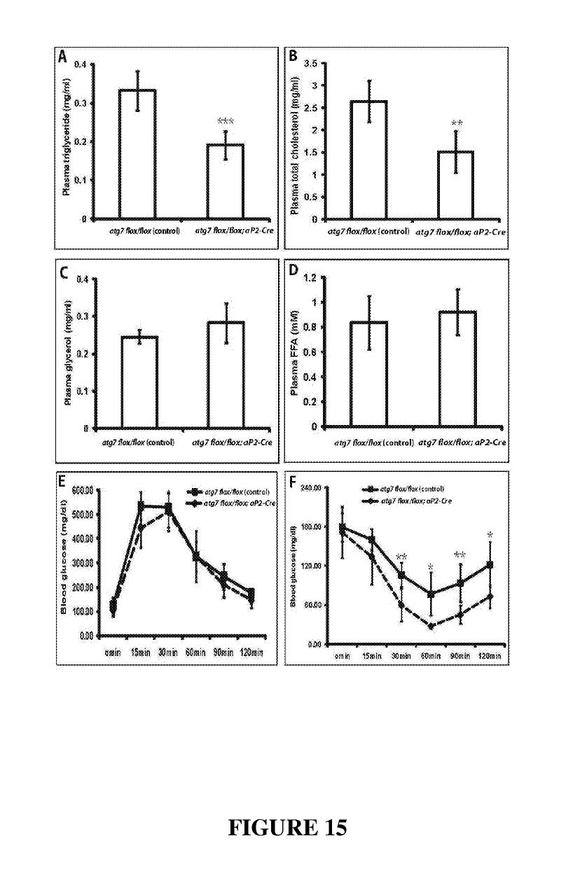

In further embodiments, autophagy inhibitors may be administered for preventing, mitigating or treating pathological conditions attributable to or associated with weight gain or obesity. One such condition is type II diabetes. Specifically, the data discussed below, and illustrated in FIG. 15F, indicates that administration of one or more autophagy inhibitors lead to alteration of adipose tissues in such a way that the subject exhibits significantly increased sensitivity to insulin. To this end, the effect of administration of an autophagy inhibitor is to counteract the insulin deficiency observed with type II diabetes.

The instant invention, however, is not limited to treating type II diabetes and may treat other conditions including, but not limited to, the following: These diseases include but not limited to the following: (1) cardiovascular diseases; (2) Hyperlipidimia; (3) Certain cancers; (4) Gallbladder disease and gallstones; (5) Osteoarthritis; (6) Gout; and (7) Breathing problems, such as sleep apnea and asthma.

Autophagy inhibitors of the present invention may be synthesized using methods known in the art or as otherwise specified herein. Unless otherwise specified, a reference to a particular compound of the present invention includes all isomeric forms of the compound, to include all diastereomers, tautomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated by reference herein. Reference to a nucleic acid or biological agent similarly refers to the specific sequences herein or otherwise known, as well as homologues thereof.

Based on the foregoing, one or more autophagy inhibitors of the present invention, either alone or in combination, may be synthesized and administered as a therapeutic composition. The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. To this end, the pharmaceutical compositions may be formulated to suit a selected route of administration, and may contain ingredients specific to the route of administration. Routes of administration of such pharmaceutical compositions are usually split into five general groups: inhaled, oral, transdermal, parenteral and suppository. In one embodiment, the pharmaceutical compositions of the present invention may be suited for parenteral administration by way of injection such as intravenous, intradermal, intramuscular, intrathecal, or subcutaneous injection. Alternatively, the composition of the present invention may be formulated for oral administration as provided herein or otherwise known in the art.

For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar or another sweetener, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including (1) the patient's age, body weight, and organ function (liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, the dose will be chosen to achieve serum levels of 1 ng/ml to 100 ng/ml with the goal of attaining effective concentrations at the target site of approximately 1 μg/ml to 10 μg/ml. Using factors such as this, a therapeutically effective amount may be administered so as to ameliorate the targeted symptoms of and/or treat or prevent obesity or diseases related thereto. Determination of a therapeutically effective amount is well within the capabilities of those skilled in the art, especially in light of the detailed disclosure and examples provided herein.

### EXAMPLES

### Example 1

### Materials and Methods for atg5−/− Test Data

### Adipocyte Differentiation of Primary MEFs

The MEFs were prepared from 13.5 days embryos of atg5+/+ and atg5−/− mice according to standard protocol. Briefly, whole mouse embryos were removed from the uterus, dissected and the head, tail, limbs and all internal organs were removed. The carcasses were minced, washed in PBS, and then incubated in 2 ml 0.05% Trypsin-EDTA (Invitrogen, CA, US) at 37° C. for 20 min with shaking. The digested cells were plated on a 100-mm dish in Dulbecco's modified Eagle's medium (DMEM, Invitrogen) with 10% fetal bovine serum (FCS, Invitrogen) and incubated at 37° C. in humidified air containing 5% CO₂. Cells were grown for 24 hr until the culture was 90% confluent, and then split and passed on. The primary MEF cells of passage three to five were treated under standard protocol to induce adipocyte differentiation (Banks, A. S., et al. Deletion of SOCS7 leads to enhanced insulin action and enlarged islets of Langerhans. J. Clin. Invest. September 2005 115, 2462-2471). Briefly, cells were seeded in 6-well plates with cover slips and propagated to confluence. 48 hours later, which was designated as Day 0, differentiation was initiated using Dulbecco's Modified Eagle's Medium (DMEM) containing 10% fetal bovine serum, 5 μg/ml insulin, 1 μM dexamethasone, 0.5 mM 3-Isobutyl-1-methylxanthine (IBMX), and 10 μM troglitazone (Sigma, MO, US). After 2-day initiation, the media was replaced with a maintenance medium (DMEM containing 10% fetal bovine serum, 5 μg/ml insulin, and 10 μM troglitazone). Fresh maintenance medium was replaced every 2 days thereafter. Cells collected at various time points, up to maximal 14 days.

Hydroxychloroquine and Chloroquine were from Thermo-Fisher and Sigma, respectively. For chloroquine treatment, the wild type primary MEFs of early passages were seeded in six-well plates. The cells were treated with 10 μM chloroquine (Sigma MO, US) at 50% confluence and propagated to full confluence. Two days after confluence, adipocyte differentiation was induced with differentiation medium containing 10 μM chloroquine. The medium was replaced with maintenance medium including 10 μM CQ two days after initiation. Fresh maintenance medium with 10 μM CQ was replaced every two days thereafter.

### Autophagosome Quantification by EM and Immunoblotting

The differentiating cells were fixed at indicated time points with 2.5% gluteraldehyde/4% paraformaldehyde in 0.1M cacodylate buffer for two hours. The samples were processed and thin sections (90 nm) were cut on a Reichert Ultracut E microtome. Sections were viewed at 80 kV with a JEOL 1200EX transmission electron microscope. Micrographs were taken in the Philips CM12 (15-20 per sample) by random sampling with a primary magnification of X6300. The cytoplasmic volume fraction of autophagic vacuoles was quantified by point counting method. Western blotting was carried out according to standard protocol. The sources of the antibodies are: MAP-LC3 antibody: made by Cocalico Biologicals (PA, US) using a recombinant rat MAP-LC3 protein as antigen; rabbit polyclonal Atg12 antibody: Cell Signaling Technology, MA; rabbit polyclonal Ran antibody: C-20, Santa Cruz, Calif., US. p62: primary antibody: guinea pig anti-p62 Cterminal specific (Cat #03-GP62-C) from American Research Products, Inc (Belmont, Mass.); secondary: donkey anti-guinea pig polyclonal antibody from Jackson ImmunoResearch Laboratories, Inc. (West Grove, Pa.).

### Imaging

Live cells were observed under the Olympus IX70 microscope with relief contrast objectives (10× or 40×). Fixed and stained cells were observed with Universal microscope Axioplan 2 imaging (Carl Zeiss, NY, US) with 20× phase contrast objectives. For time-lapse microscopy, cells were plated on tissue culture plates and monitored using Olympus IX70 microscope with the 37° C. and 5% CO₂ environmental chamber using 10× objectives. Images were collected with CCD video camera (Model MicroMax; Princeton Instruments, Trenton, N.J., US) at 5 min intervals and saved as images stacks using IPLab software (BD Biosciences Bioimaging, Rockville, Md., US). Images were processed using Image J software (NIH, Bethesda, Md., US).

### Lipid Droplet Staining

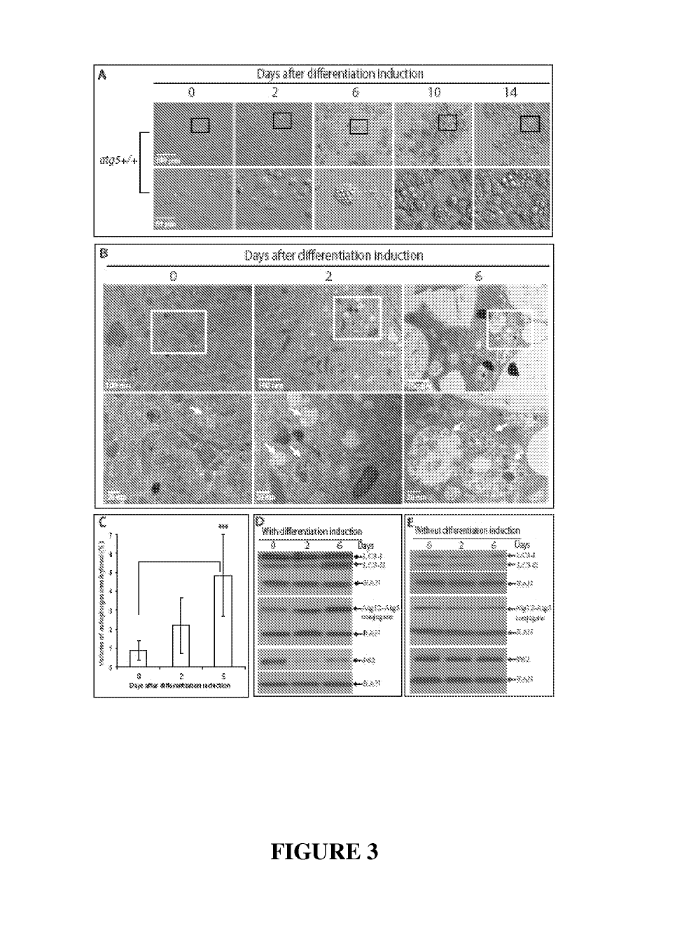

Bodipy: The differentiating cells were stained with BODIPY 493/503 (Invitrogen, CA, US) as described (DiDanato, D. et al. Fixation methods for the study of lipid droplets by immunofluorescence microscopy. J Histochem. Cytochem June 2003 51, 773-780; Gocze, P. M. et al. Factors underlying the variability of lipid droplet fluorescence in MA-10 Lydig tumor cells. Cytometry 17, 151-158.) Briefly, the cell culture slides were washed once with phosphate-buffered saline (PBS), fixed with 3% paraformaldehyde in PBS for 30 minutes at room temperature, and washed 4 times with PBS, and then stained with 10 μg/ml Bodipy 493/503 at room temperature for 15 minutes in darkness, mounted with Vectashield Mounting Medium (Vector Laboratories, CA, US). Oil Red-O: The cells on cover slides were stained with Oil Red-O (Sigma, MO, US) according to Kim et al with some modification (Kim, Y. K., et al. Reversine stimulates adipocyte differentiation and downregulates Akt and p70(s6k) singaling pathways in 3T3-L1 cells. Biochem Biophys Res Commun 358, 553-558). Oil Red-O was dissolved in isopropanol to 3.5 mg/ml. Before using, dilute 6 parts Oil Red-O stock with 4 parts H₂O, sit at room temp for 20 min, and filter through 0.2 μm filter. The slides were washed once with PBS, fixed with 4% paraformaldehyde (Fisher Scientific, PA, US) buffered with PBS for 1 hr, and then washed twice with 60% isopropanol for 5 minutes each. Cells were then air dried and stained with Oil Red-O working solution for 30 minutes at 25° C. For FIG. 3C, slides were counterstained with hematoxylin for 1 min. To quantify staining (FIG. 3D), Oil Red-O were extracted from cells on the slides with isopropanol containing 4% NP-40, optical density (OD) was measured at wavelength of 520 nm.

cDNA Amplification and Quantitative Real-Time PCR:

RNA was extracted from cell culture lysates using TRIzol reagent (Invitrogen, Carlsbad, Calif.) according to standard protocol. Subsequent RNA quality assessment, cDNA amplification, and quantitative RT-PCR reactions were carried out by the Bionomics Research and Technology Center (BRTC) of the Environmental and Occupational Health Science Institute (EOSHI) at Rutgers University, Piscataway, N.J. (detailed protocols available at http://eohsi-brtc.com). RNA quality was assessed by electrophoresis using the Agilent Bioanalyzer 2100 (Agilent Technologies, Santa Clara, Calif.) and by spectrophotometric analysis prior to cDNA synthesis. Between 5 and 20 ng of total RNA from each sample was used to generate high fidelity cDNA for quantitative PCR. The Ribo-SPIA (NuGen, San Carlos, Calif.) linear amplification process was used to generate “antisense” cDNA. The SPIA process (NuGen, San Carlos, Calif.) was used to amplify cDNA and a 1:200 dilution of amplified cDNA product was used for subsequent QPCR analysis.

Six genes relevant to adipocyte differentiation were selected for quantitative realtime PCR analysis to validate the findings of the microarray analysis. Gpam, Cebpa, Pparg, Fabp4, Agpat2, and Plin were identified as candidate genes for analysis and two further genes, Ube23DNORM and Wbp11NORM, were selected as normalizers for the PCR reactions. Gene expression was examined using Taqman chemistry with probes and primers designed using the Roche Universal Probe Library design algorithm (www.universalprobelibrary.com). Results of the probe design are as follows: Gpam, left primer GAGGCAAGGACATTTATGTGG (SEQ ID NO: 1) and right primer GGTGCTTTCACAATCACTCG (SEQ ID NO: 2); Cebpa, left primer CTGGCTCTGGGTCTGGAA (SEQ ID NO: 3) and right primer AGCCACAGGGGTGTGTGTA (SEQ ID NO: 4); Pparg, left primer CTCTCAGCTGTTCGCCAAG (SEQ ID NO: 5) and right primer CACGTGCTCTGTGACGATCT (SEQ ID NO: 6); Fabp4, left primer GCACGGTCTCTCTGCAATC (SEQ ID NO: 7) and right primer ACAATCAATCAGCGCAGGA (SEQ ID NO: 8); Ucp1, left primer CCAGTGGATGTGGTAAAAACAA (SEQ ID NO: 9) and right primer CACAGCTTGGTACGCTTGG (SEQ ID NO: 10); Agpat2, left primer TTCCCACCTCAAGCCTGT (SEQ ID NO: 11) and right primer TGCCTTGTGGTCTTGTGG (SEQ ID NO: 12); Plin, left primer CTCCGGCCTTTCCTCTCTA (SEQ ID NO: 13) and right primer GGGGGAGTGATGACATGG (SEQ ID NO: 14); Ube23NORM, left primer TTAGTGATTTGGCCCGTGA (SEQ ID NO: 15) and right primer TGGCTTGCCAATGAAACAT (SEQ ID NO: 16); and Wbp11NORM, left primer GAGCAATGTCCACTGTCAGG (SEQ ID NO: 17) and right primer ATCCCAGCAGGCAAACAT (SEQ ID NO: 18). The following dye combinations for probe generation were used for detection and data normalization: FAM (for the genes of interest), HEX (for normalizer genes) and BHQ1 (non-fluorescent quencher) and ROX (reference). All probes were 8 mer MGB probes selected from the Roche Universal Probe Library as appointed per each assay design. Prior to comparative analysis, a validation experiment was performed in order to determine the relative efficiency of the assays designed for the genes of interest and Wbp11NORM and Ube23DNORM which were subsequently used as reference genes for comparative analysis. All reactions were performed in triplicate and the experiments were replicated three times. All reactions were run in an ABI 7900 (Applied Biosystems, Foster City, Calif.) with the following cycle parameters: 1 cycle of 50° C. (2 min) followed by 95° C. (10 min.), 40 cycles of 95° C. (15 sec) followed by 60° C. (1 min). Data was collected at every temperature phase during every cycle and analyzed using the Sequence Detection Software (Applied Biosystems, Foster City Calif.) while relative quantitation using the comparative threshold cycle (CT) method was performed. The expression levels of the genes of interest were presented as the relative levels to the mRNA level of the control gene Wbp11NORM.

### TUNEL Assay

The differentiating cells growing on cover slides were fixed with PBS buffered formalin for 15 min. The TUNEL assay was conducted with In Situ Cell Death Detection Kit, TMR red (Roche Applied Science, IN, US) according to the instruction of the manufacturer. The slides were counter-stained with BODIPY 493/503 (10 μg/ml) and DAPI (1 μg/ml) for 10 min before mounting and taking picture under the microscope. In randomly selected areas, the differentiating cell with Bodipy 493/503 signal (green) were counted and cells with TUNEL positive signal (red) were counted among these cells. The percentage of apoptotic cells among differentiating cells were calculated. For chloroquine treatment experiments, the apoptotic cell populations were quantified by Coulter Cytomics FC500 Flow Cytometer (Beckman Coulter, CA, US).

### In Vivo Immunofluorescence Analyses

Neonatal mice (within 12 hr after birth) were sacrificed, fixed with 4% paraformaldehyde buffered with PBS, and embedded in paraffin. After genotyping with PCR method, the transverse sections were cut at the level of scapulae. Immunofluorescence was performed with standard protocol. Briefly, sections were deparaffinized with Xylene and rehydrated through graded ethanol. Antigen unmasking was carried out in 10 mM sodium citrate buffer (pH 6.0) at 95-99° C. for 10 min. Slides were allowed to cool down at room temperature for 30 min and rinsed with H₂O and PBS. Specimens were blocked with 5% goat serum in PBS/Triton for 1 hr, followed by incubating with Perilipin A antibody (Sigma, MO, US, 1:50 dilution) at 4° C. overnight. For immunostaining detection, slides were incubated with secondary antibody FITC-Goat Anti-Rabbit IgG (Invitrogen, CA, US, 1:100 dilution) for 1 hr, rinsed with PBS, and mounted with Vectashield Mounting Medium (Vector Lab. Inc., CA, US). Pictures were taken with a Universal Microscope Axioplan 2 imaging system (Carl Zeiss, NY, US) with 100× phase contrast objectives. The diameters of lipid droplet in the pictures were measured with Adobe Photoshop software (Adobe Systems, Inc, San Jose, Calif.).

### Antibodies

Western blotting was carried out with standard protocol. The sources of the antibodies are: MAP-LC3 antibody was made by Cocalico Biologicals (PA, US) using a recombinant rat MAP-LC3 protein as antigen; rabbit polyclonal Atg12 antibody (Cell Signaling technology, MA), rabbit polyclonal Beclin 1 antibody (BECN1, H-300, Santa Cruz, Calif.), rabbit polyclonal Perilipin A antibody (Sigma, MO, US), rabbit polyclonal Ran antibody (C-20, Santa Cruz, Calif., US).

### Autophagosome Quantification

The differentiating cells were fixed at various time points with 2.5% gluteraldehyde/4% paraformaldehyde in 0.1M cacodylate buffer for 2 hours. The samples were processed and thin sections (90 nm) were cut on a Reichert Ultracut E microtome. Sections were viewed at 80 kV with a JEOL 1200EX transmission electron microscope. Micrographs were taken in the Philips CM12 (15-20 per sample) by random sampling with a primary magnification of X6300. We estimated the cytoplasmic volume fraction of autophagic vacuoles by point counting.

### Example 2

### Autophagy was Activated in Wild Type MEFs During Adipocyte Differentiation

The activation of autophagy in the primary MEFs during adipogenesis via morphology study was analyzed with electron microscopy (EM) as well as by molecular characterization with autophagy-specific markers. Similar to the induction protocol of adipogenesis in 3T3-L1 cells, the primary MEFs were first grown to confluence. Two days after confluence, a cocktail of differentiation agents containing dexamethasone (DEX)/3-Isobutyl-1-methylxanthine (IBMX)/troglitazone/insulins, was added to the medium to induce differentiation and the time was recorded as Day 0 of induction. Two days later (or on Day 2 of induction), the differentiation maintenance medium (containing only insulin and troglitazone) replaced the original differentiation cocktail. From then on, the fresh maintenance medium was added to the cells every two days to replace the old medium. The differentiation of cells was monitored with a microscope equipped with relief contrast lens, which was used to observe the three dimensional structure of the cells. As shown in FIG. 3A, the kinetics of adipogenesis in wild type primary MEFs was very similar to that of 3T3-L1 cells: on Day 2 of induction, isolated cells started to “inflate” to form a spheroid morphology from the original flat morphology, and micro-size lipid droplets started to accumulate in the spheroid cells; on Day 6 of differentiation induction, small patches of the spheroid cells formed, each cell in the patch containing many small lipid droplets; as differentiation continued, more flat cells participated in differentiation and exhibited the “inflated” spheroid morphology; in the meantime small lipid droplets grew larger in size or fused with each other; on Day 14, the majority of cells formed patches of “inflated” spheroid cells, many of which contained one or several large lipid droplets.

An electron microscopy was performed to analyze autophagosome formation during adipogenesis of the wild type primary MEFs. As shown in FIGS. 3B and 3C, ultrasturcture of cells on Day 0, Day 2, and Day 6 during differentiation induction were analyzed by EM and the volume of autophagosomes was quantified. Prior to induction of differentiation (Day 0), autophagosomes occupied about 1% of the cytoplasmic volume. The level of autophagosomes steadily increased as the cells underwent adipogenesis. By Day 6, around 5% of the total cytoplasmic volume (volume of lipid droplets included) was occupied by autophagosomes.

Autophagy activation during adipogenesis was further analyzed with specific autophagy molecular markers. MAP-LC3 is a mammalian homolog of the yeast Atg8 protein. During autophagy activation, it is cleaved at its C-terminus and the N-terminus portion of MAP-LC3 is conjugated with phospholipids and translocated onto the autophagosome. The abundance of the processed form of MAP-LC3, known as LC3-II, reflects a steady-state level of autophagosomes. As shown in FIG. 3D (upper panel), the level of LC3-II dramatically increased as differentiation progressed. To confirm that the increase of autophagosomes also reflected the increase of functional autophagic degradation, autophagy flux was analyzed by measuring the levels of the p62, which is a common autophagosome cargo whose degradation reflects the levels of autophagy flux. As shown in FIG. 3D, lower panel, induction of adipogenesis drastically reduced p62 levels, indicating an increase of autophagy flux.

In addition, the levels of other proteins that are specifically involved in autophagy was measured. Interestingly, the level of the Atg5-Atg12 protein conjugate also increased significantly during adipogenesis (FIG. 3D, middle panel). Importantly, the increase of autophagosomes formation, autophagy flux, and Atg5-Atg12 conjugation were specifically associated with adipogenesis. As a control, when the MEFs were not induced for adipogenesis, LC3-II levels slightly reduced over time, and ATG5-ATG12 conjugate levels and p62 levels remained unchanged, as shown in FIG. 1E. Taken together, these results demonstrated that autophagic activity was increased in the primary MEFs undergoing adipogenesis.

### Example 3

### Autophagy Deficient Primary Atg5−/− MEFs Exhibited Significantly Reduced Efficiency in Adipogenesis

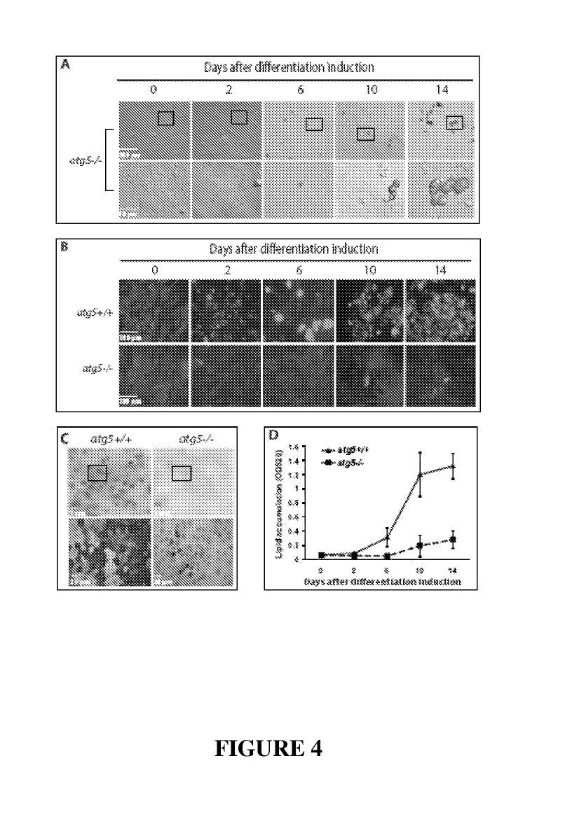

A functional role of autophagy in adipocyte differentiation was studied by examining the impact of atg5 deletion on adipogenesis in the primary MEF model. Mice with homozygous atg5 deletion (atg5−/−) develop without any apparent defects and are born in normal Mendelian ratios, but die within the first day following birth in part due to failure to cope with neonatal starvation. Measurements of autophagy demonstrated that the formation of autophagosomes was absent in the tissues of the atg5−/− mice. The wild type and atg5−/− primary MEFs from E13.5 embryos of the same pregnant mother were induced for adipogenesis. The progression of differentiation was monitored by microscopy with relief contrast lens. In contrast to wild type (atg5+/+) MEFs, which underwent normal adipogenesis as shown in FIG. 3A, atg5−/− cells initially accumulated small lipid droplets but seemed to become inert after the initiation phase of differentiation (FIG. 4A). These cells with small lipid droplets had difficulty progressing to more advanced stages. At any given time within the 14-day differentiation, only a small portion of atg5−/− cells showed initial morphologic changes with the spheroid morphology and with the accumulation of micro-size to small size lipid droplets. Even at the end of the 14-day differentiation, very few cells were found in the more advanced differentiation stages signified by one or a few large lipid droplets.

Lipid accumulation is a hallmark of adipocyte differentiation. In addition to the morphological characterization of adipocyte differentiation, lipid accumulation in the atg5−/− cells was analyzed and their wild type counterparts following induction of differentiation. As shown in FIG. 4B, cells at various time points post-differentiation induction were fixed and stained with Bodipy 493/503, a fluorescent dye that specifically stains intracellular lipid droplets. As the differentiation progressed, more and more lipid staining was observed in the wild type cells. In contrast, very limited lipid staining in the atg5−/− cells was observed even on day 14, when the majority of wild type cells were in advanced differentiation stages. The differences of lipid accumulation between the two types of MEFs were quantified using Oil Red-O, another dye that specifically stains the lipid droplets and can be extracted for spectrometry measurement. As shown in FIG. 4C, at the end of 14 days of differentiation, a very dramatic difference in Oil Red-O staining between the wild type and the mutant cells was observed. At various time points Oil Red-O staining was performed and the dye was extracted from the cells for quantification by spectrometry. As summarized in FIG. 4D, the atg5−/− MEFs exhibited a significant defect in accumulating lipid droplets upon differentiation induction, which was completely consistent with the morphological analysis (FIGS. 3A and 4A).

The adipogenesis defects observed in these atg5−/− cells were not clone-specific effects. The same phenotypes were observed in four pairs of MEFs from three independent pairs of breeding parents. The primary MEFs of early passages (passages three to five) were used in the adipocyte differentiation experiments. MEFs of earlier passages (passages one to two) was also tested, as well as MEFs of later passages (passages six to eight). While clear differences in adipocyte differentiation were observed between the wild type cells and the atg5−/− cells of all passages, interestingly, it appeared that the inhibitory effect of autophagy deficiency on adipogenesis was less dramatic in the earlier passage cells than the later passage cells (data not shown). These results suggest that events secondary to autophagy deficiency may accumulate over time which can exacerbate the direct effect of autophagy deficiency on adipocyte differentiation.

### Example 4

### Gene Expression Analysis of the Differentiating Wild Type and Atg5−/− MEFs

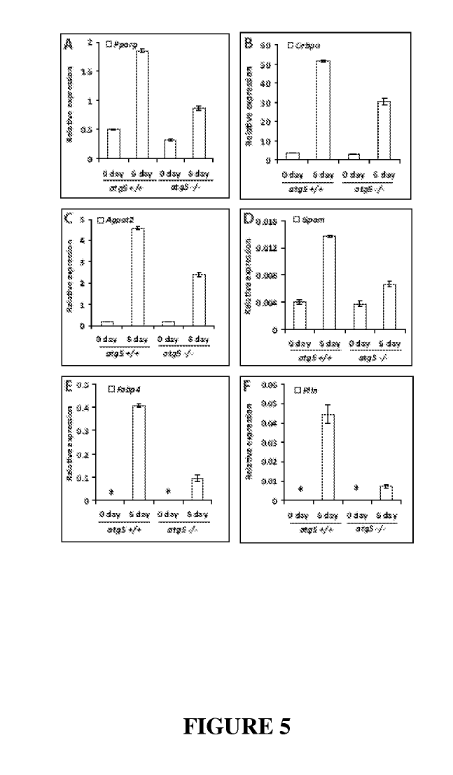

The mRNA levels of genes in cells prior to differentiation induction were compared to those in cells six days after differentiation induction in wild type and atg5−/− MEFs. First, gene expression profiling experiments with oligonucleotide microarray was performed. The genes involved in adipocyte differentiation exhibited the most dramatic changes upon differentiation induction in both wild type and atg5−/− cells (data not shown). The expression levels of a subset of those genes were confirmed by quantitative PCR analysis, as shown in FIG. 5. It seemed that most genes that were upregulated in the wild type cells were also upregulated in the atg5−/− cells; however, the extent of gene activation was less robust in the atg5−/− cells. Moreover, it appeared that the later transcriptional events of adipogenesis, such as upregulation of Fabp4 and Perilipin genes, were more severely impacted in the atg5−/− cells as compared to the early transcriptional events, such as PPARγ and CEBPα upregulation. Although these results did not pinpoint a particular event in which autophagy interacted with adipogenesis, they did confirm at the molecular level that less atg5−/− cells underwent adipogenesis.

### Example 5

### Time-Lapse Microscopy Showed that Adipocyte Differentiation Stalls at an Early Stage in Primary Atg5−/− MEFs

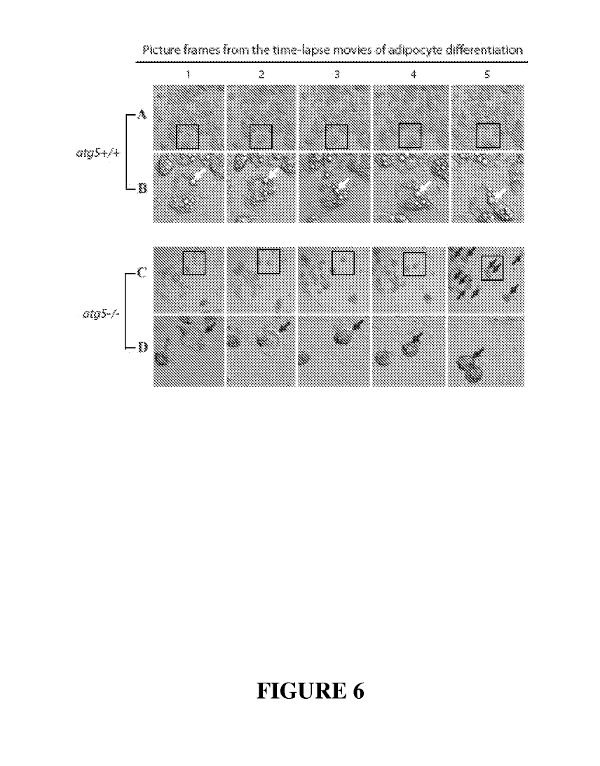

To determine the stages at which adipocyte differentiation was affected and the likely cause of the defect, movies of actively differentiating atg5+/+ and atg5−/− MEFs was generated using time-lapse microscopy. FIGS. 6A and 6B show picture frames selected from two-day movies of differentiating wild type cells starting on day three. Small lipid droplets emerged and actively fused and consolidated into larger droplets during this period of time. FIGS. 6C and 6D are picture frames from movies of atg5−/− MEFs in the same time frame as the wild type cells shown in FIGS. 4A and 4B. In contrast to the differentiating wild type cells, most differentiating atg5−/− cells at the beginning of the movie (day three) had accumulated only numerous micro lipid droplets. Strikingly, most of these differentiating atg5−/− cells stalled at this stage and were unable to progress further morphologically. Eventually these cells slowly lost their anchorage to neighboring cells, began to rotate freely in the medium and ultimately died (FIGS. 6C and 6D). It is noteworthy in the movies that only the atg5−/− cells that underwent initial early differentiation died, while the undifferentiated cells remained normal and alive. These results suggest that the atg5 function might not be indispensable for adipogenesis initiation in accumulating micro-sized lipid droplets but it was critical for efficient progression of adipogenesis. As a result, atg5 deletion frequently led to aborted differentiation.

### Example 6

### The Differentiating Atg5−/− Cells Exhibited Higher Levels of Apoptosis than the Wild Type Cells

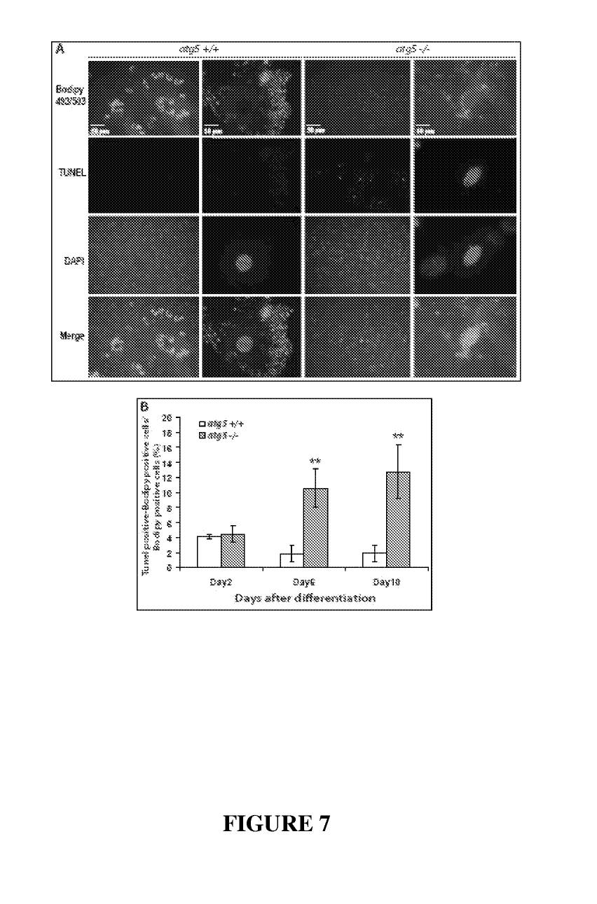

To determine whether the aborted cells during differentiation died of apoptosis, TUNEL assays were performed, which detect DNA breakage/fragmention, a hallmark of apoptosis. As shown in FIG. 7, the wild type and atg5−/− primary MEFs were analyzed at various time points during adipogenesis by TUNEL assay. These cells were also costained with Bodipy 493/503 to monitor adipogenesis. Interestingly, essentially all TUNEL positive cells were also Bodipy 493/503 positive (FIG. 7A), indicating that only the cells that started adipogenesis and began accumulating lipid droplets were vulnerable to apoptosis. For the wild type cells, at early stages of differentiation (Day 2), there was a small percentage of the differentiating cells underwent apoptosis as shown in FIG. 7B. As differentiation progressed, the cells undergoing apoptosis decreased. In contrast, the percentage of the differentiating atg5−/− cells undergoing apoptosis at Day 6 was significantly higher than that of the wild type cells. Moreover, the apoptotic cells as a percentage of the differentiating cells continued to increase in later time points in the atg5−/− cell. These observations were consistent with FIG. 6 and suggested that in the cellular adipogenesis model system the differentiating cells that failed to mature would eventually die of apoptosis.

### Example 7

### The Atg5−/− Late-Stage Embryos and Neonatal Pups had Less Subcutaneous Fat Cells

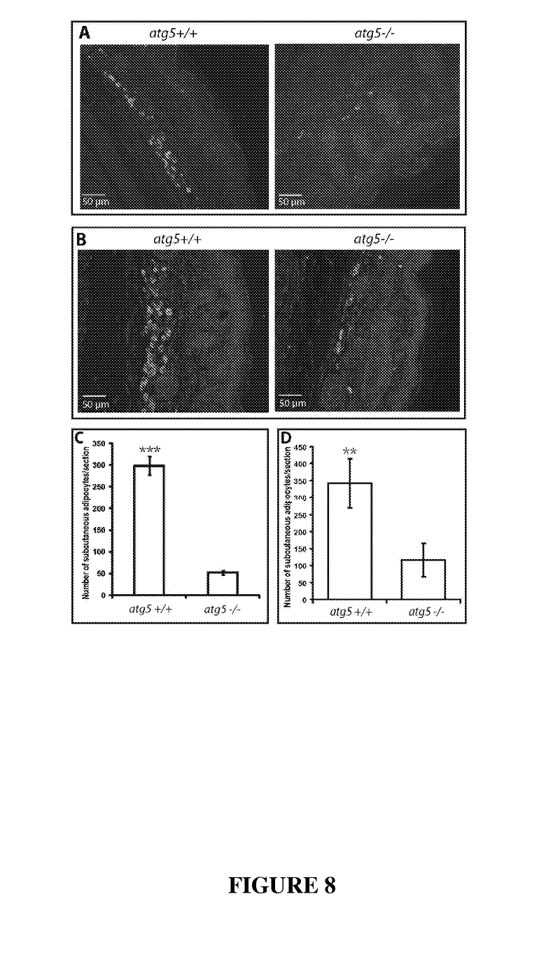

To determine whether atg5 deletion affects adipogenesis in vivo, the adipocytes were analyzed in the atg5−/− late-stage embryos and neonatal pups and their wild type counterparts. As described earlier, the atg5−/− mice develop normally throughout gestation, but always die within the first day of birth partly due to the fact that these mice cannot mobilize sufficient internal nutrients to survive the neonatal starvation period. The adipocytes of the E18.5 atg5+/+ and atg5−/− embryos were analyzed, as well as pups within 12 hours after birth. In rodents, the white fat tissue at birth is not well developed; however, dispersed white adipocytes can be observed in subcutaneous regions. Transverse sections of the mice were cut at the level of the scapulae, where the subcutaneous white adipocytes can be analyzed. The tissues were examined by immunofluorescence microscopy with antibody against perilipin A, a protein that localizes on the membrane of lipid droplets of adipocytes. FIG. 8A shows the immunostaining of tissues in subcutaneous regions of the embryos. The atg5−/− embryos had only 15% of perilipin A positive adipocytes at corresponding subcutaneous regions compared to their wild type counterparts (FIGS. 8A and 8C), suggesting adipogenesis of white adipocytes was significantly reduced. Similarly, the neonatal atg5−/− pups had drastically reduced perilipin A positive adipocytes at corresponding subcutaneous regions compared to their wild type littermates (FIGS. 8B and 8D). Together, these results suggest that atg5 deletion affects adipogenesis in vivo.

### Example 8

### Autophagy Inhibitor Chloroquine Blocked Adipocyte Differentiation in Primary MEFs

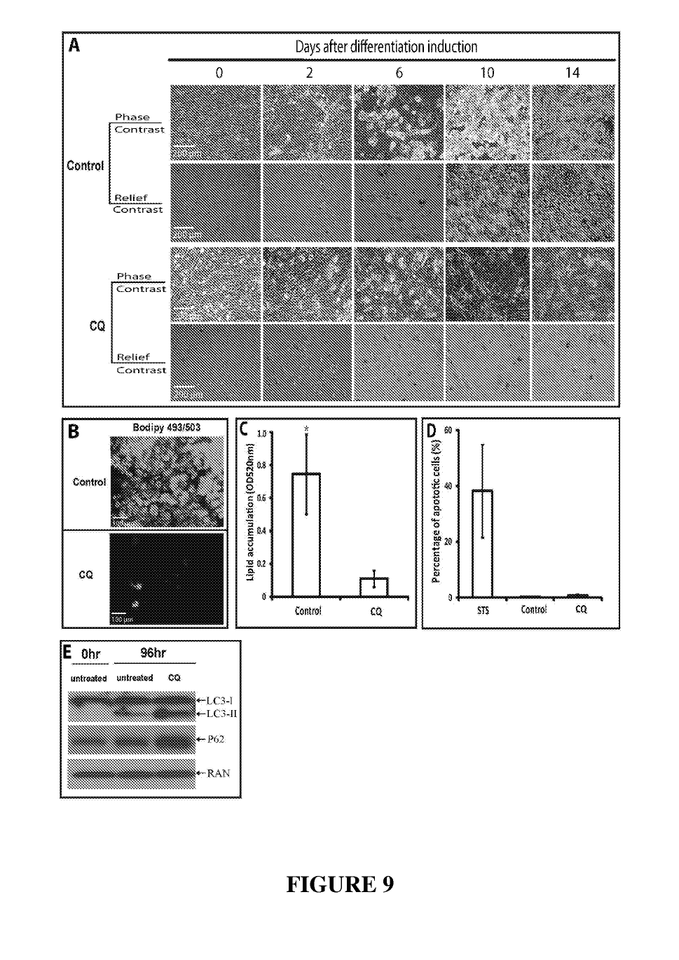

Chloroquine, a FDA approved drug for malaria and rheumatoid arthritis, targets the lysosome and it also blocks the fusion of the autophagosome with the lysosome. Chloroquine is an effective autophagy inhibitor which has been extensively used for autophagy inhibition both in clinical trials and in laboratory studies. The effect of chloroquine on the adipocyte differentiation of primary MEFs was determined. The wild type primary MEFs were treated to induce adipogenesis with or without the presence of μM chloroquine, a concentration that inhibits the fusion of the autophagosome with lysosome (thereby increases intracellular levels of autophagosomes but inhibits autophagy flux (FIG. 9E)). At this concentration chloroquine had little cytotoxic effect on the MEFs (FIG. 9D). As shown in FIG. 9A, chloroquine significantly inhibited normal adipocyte differentiation as detected by morphological analysis. Consistently, lipid accumulation analysis using Bodipy 493/503 (FIG. 9B) or Oil Red-O (FIG. 9C) staining showed the same inhibitory effect of chloroquine on adipocyte differentiation. Together, these results indicate that chloroquine effectively inhibits adipogenesis in the primary MEF model.

### Example 9

### Materials and Methods for Atg7−/− Test Data

Generation and Characterization of Adipose-Specific atg7 Knockout Mice

The atg7flox/flox mice (16) and aP2 (Fabp4)-Cre transgenic mice, obtained from The Jackson Laboratory, ME were crossed to produce the adipose tissue-specific atg7 conditional knockout mice atg7flox/flox; aP2-Cre. The genotypes of the mice were determined and deletion of atg7 in adipose tissue were confirmed with PCR using primers described previously. The body weights of were measured once every week after week 4. At the age of 18˜20 weeks, mice were sacrificed for tissue/organ analysis. Food intake experiments were performed with mice housed individually in metabolic cages (Nalgene, NY). For high-fat diet (HFD) experiment, mice were fed with HFD (60 kcal % fat, Research Diets, NJ) at the age of 8 weeks for 8 weeks, body weight were measured once every week.

### Western Blotting, Tissue Analyses, and EM Analyses

Western blotting was carried out according to standard protocol. The sources of the antibodies are: rabbit polyclonal Atg12 antibody (Cell Signaling Technology, MA), rabbit polyclonal Atg7 antibody (Cell Signaling Technology, MA), rabbit polyclonal Atg3 antibody (Abgene, CA), rabbit polyclonal Ran antibody (C-20, Santa Cruz, Calif.), rabbit polyclonal Perilipin A antibody (Sigma, MO), rabbit polyclonal UCP1 antibody (Abcam Inc, MA). Mice fat tissues were fixed with 4% paraformaldehyde buffered with PBS, and embedded in paraffin. Slides were stained with hematoxylin and eosin (H & E) for histological analysis. Immunofluorescence was performed on paraffin-embedded sections according to standard protocol. The source of antibodies: Perilipin A antibody (Sigma, MO, 1:50 dilution), COXII antibody (Cayman Chemical, Mi, 1:50 dilution), secondary antibody FITC-Goat Anti-Rabbit IgG (Invitrogen, CA, 1:100 dilution). In some experiments, 100 ng/ml DAPI was added to the secondary antibody solution to co-stain the nuclei. Pictures were taken with a Universal Microscope Axioplan 2 imaging system (Carl Zeiss, NY) with 100× phase contrast objectives.

For cell size quantification, pictures of the H & E staining slides were taken. The area of the picture (S) and the total number of cells (nuclei, N) in the picture were then determined. The average radius (r) of the cells was calculated as the square root of S/(pi*N) and the average volume was determined. For each data point, six random pictures were used. For quantification of the size of lipid droplets, the diameters of lipid droplets in the pictures were measured with Adobe Photoshop software (Adobe Systems, Inc, San Jose, Calif.) and the total volume of lipid droplets was calculated. For each data point, fifty random lipid droplets in a representative picture were measured and calculated.

For EM analysis, the white fat tissue (gonadal fat pad) and brown fat tissue (interscapular fat pad) were fixed with 2.5% gluteraldehyde/4% paraformaldehyde in 0.1M cacodylate buffer for two hours. The samples were analyzed as previously described above.

### Adipocyte Differentiation of Primary MEFs and Lipid Staining

The MEFs were prepared from 13.5 day atg7+/+ and atg7−/− embryos according to standard protocol. The primary MEFs of passages three through five were induced for adipocyte differentiation, as noted above, and the differentiating cells were subjected to lipid staining.

### Plasma Lipid Measurement, Glucose and Insulin Tolerance Tests

Blood samples were collected from the tail of the mice fasted overnight with heparinized micro-hematocrit capillary tubes (Fisher, PA). Plasma was obtained by centrifuge the blood samples with Readacrit centrifuge (Clay Adams, NJ) for 3 min. Plasma glycerol and triglycerides were measured with a serum triglyceride determination kit (Sigma, MO). Total cholesterols in plasma were measured with a Total Cholesterol/Cholesteryl Ester Quantification Kit (BioVision, CA). Plasma free fatty acids were measured with Free Fatty Acids, Half-Micro Test Kit (Roche, IN).

For glucose tolerance tests, mice were fasted overnight and intraperitoneally injected with 20% glucose at a dose of 2 g/kg body weight. Blood was obtained from the tail at time points 0, 15, 30, 60, 90, and 120 min for glucose measurement using an OneTouch UltraSmart Blood Glucose Monitoring System (Lifescan, CA). For insulin tolerance tests, mice were fasted for 5 hr and intraperitoneally injected with 0.75 U/kg body weight recombinant human insulin (Eli Lilly and Company, IN). Blood was obtained from the tail at time points 0, 15, 30, 60, 90, and 120 min for glucose measurement using the same blood sugar monitoring system.

### Example 10

### Adipose-Specific Atg7 Knockout Mice had Drastically Reduced White Fat Mass and Reduced Body Weight

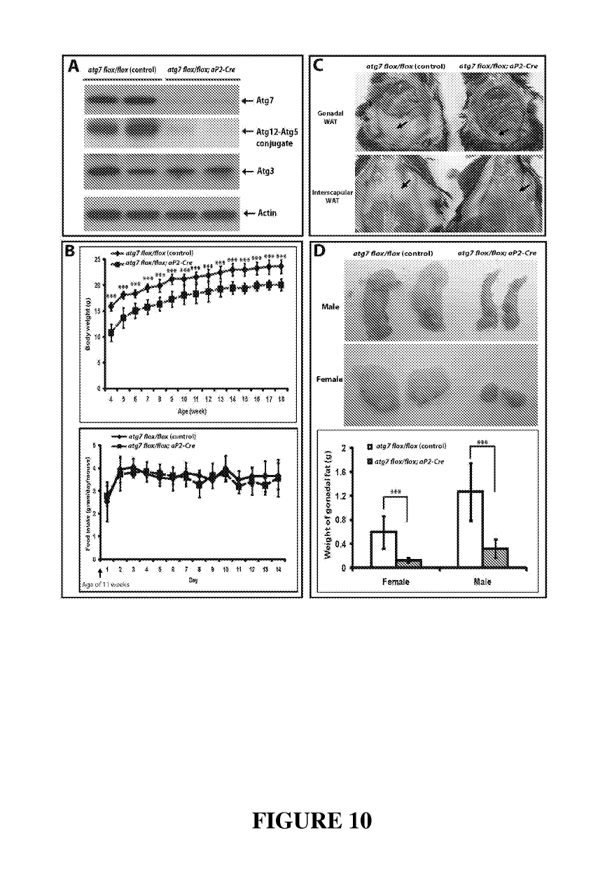

Adipose-specific atg7 knockout mice were generated by crossing flox-atg7 mice with aP2-cre mice, in which CRE expression is under the control of an adipose tissue specific aP2 (Fatty Acid Binding Protein 4, FABP4) promoter, which is active in both white and brown fat tissues. The homozygous flox-atg7/aP2-cre F2 mice were born in normal Mendelian ratios, indicating that the deletion of the atg7 gene in adipose tissues did not interfere with embryonic development and survival of the fetus. The ablation of atg7 expression in white fat tissues was nearly complete, as confirmed by immunoblotting analysis shown in FIG. 10A. In addition, the levels of Atg5-Atg12 conjugates were almost undetectable (FIG. 10A), which was indicative of autophagy deficiency in these tissues. As they grew, the atg7 conditional knockout mice were visibly smaller and shivered more frequently than their control atg7 wild type littermates, but otherwise appeared normal. Both the male and female homozygous atg7 conditional knockout mice appeared to be infertile and failed to produce any offspring.

Body weight was compared between the atg7 knockout mice and their littermates after weaning (at 3 weeks of age). The upper panel of FIG. 10B shows the body weight chart of the female mice. The average body weight of the atg7 adipose-specific knockout mice was around 12 grams at the age of 4 weeks vs. around 16 grams in the control atg7 wild type mice. The difference in body weight was maintained and found to be statistically significant through 18 weeks of age when the experiment was stopped. Similar results were obtained with the male mice (data not shown). Interestingly, the total food intake rates (per animal) were almost identical between the atg7 knockout and control mice, as shown in FIG. 10B (lower panel), suggesting either a reduced efficiency in energy storage or an increased energy expenditure rate, or both, in the atg7 conditional knockout mice.

The fat tissues in the mice were analyzed. FIG. 10C shows the gross appearance of gonadal fat pads as well as white fat tissue in scapular region, in which a striking reduction of fat mass in the atg7 conditional knockout mice was evident. The white adipose tissues in other regions of the mutant mice, including retroperitoneal fat and inguinal fat deposits, showed a similar extent of reduction in mass (data not shown). The gonadal fat pad in abdominal cavity, the largest and the most easily dissected adipose depot in mouse, comprises about 30% of all fat mass and serves as a standard quantitative measurement for white fat mass. FIG. 10D shows that the gonadal fat pads of the atg7 conditional knockout mice (uterine fat in female and epididymal fat in male) were typically 15% of the mass of those found in the control atg7 wild type littermates.

Importantly, other organs in the atg7 conditional knockout mice did not appear to have any defects and the weight of liver, heart, lung, kidney, and brain did not exhibit any significant difference from those in the control atg7 wild type mice (data not shown). Together, these results reveal that deletion of the atg7 gene in adipose tissue has a profound impact on the mass of white adipose tissue deposits in adult mice.

### Example 11

### Atg7 Knockout White Adipose Tissues Contained Smaller Adipocytes and had Large Populations of Multilocular Cells with Significant Amounts of Cytoplasm, but Exhibited no Apparent Changes in Adipocyte-Specific Gene Expression

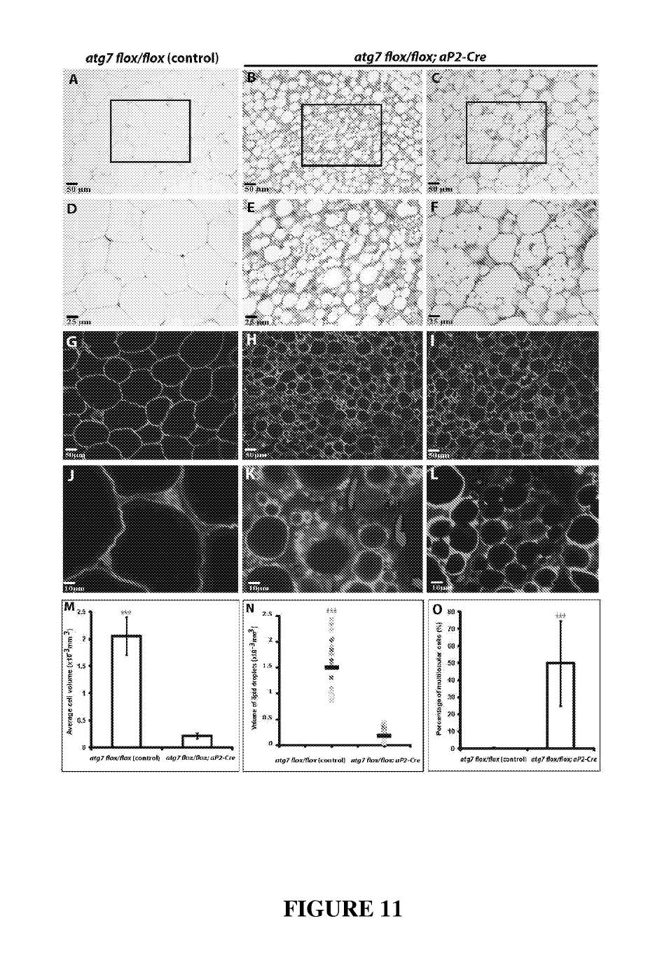

Histological analysis of gonadal fat was performed. FIG. 11 shows the results of uterine fat pad analysis from representative female mice. Hematoxylin and eosin staining of tissues showed that control atg7 wild type white adipose tissue (FIGS. 11A and 11D) was morphologically homogeneous and exhibited typical structure in which almost the whole cell was occupied by one large lipid droplet while cytoplasm was essentially undetectable. In contrast, the atg7 knockout white adipose tissue samples were heterogeneous (FIGS. 11B-C and 11E-F). The mutant cells was smaller (FIG. 11M) and a large population of the cells (around 50%) contained significant amount of cytoplasm (FIGS. 11B-C and 11E-F, stained in red). Immunofluorescence microscopy was performed with perilipin antibody, which labels the membrane of the lipid droplets in the cells (FIG. 11G-11L). While all the wild type adipocytes were unilocular (containing only one lipid droplet) (FIGS. 11G and 11J), around 50% of the atg7 knockout adipocytes were multilocular (containing multiple lipid droplets) (FIGS. 11H-I, 11K-L, 11O). On average, the size of the lipid droplets in the mutant adipocytes was smaller (FIG. 11H-I, 11K-L, 11N). Similar results were obtained from the epididymal fat pad analysis of male mice (data not shown).

To investigate whether atg7 deletion had an impact on the expression of the important adipocyte-related genes, including gpam, cebpa, pparg, fabp4, ucp1, agpat2, and plin, quantitative PCR was performed to compare the mRNA levels of these genes. There was little change in the expression pattern of these genes between the atg7 knockout white fat and the wild type control. It was noteworthy that although the atg7 knockout white adipose tissue gained a number of phenotypical features of brown fat, including multilocular lipid droplets, increased cytoplasmic volume and enriched mitochondria content, it did not show a significant increase in expression of these genes when compared to wild-type tissue. Furthermore, the expression of ucp1 in the mutant adipose tissue was negligible as compared to that found in wild-type brown fat control samples. Together, these results suggest that Atg7 may play a critical role in the elimination of cytoplasm that is presumably required for the formation and/or expansion of large, unilocular lipid droplets in WAT.

### Example 12

### Atg7 Knockout White Adipose Tissues had Increased Mitochondria Content

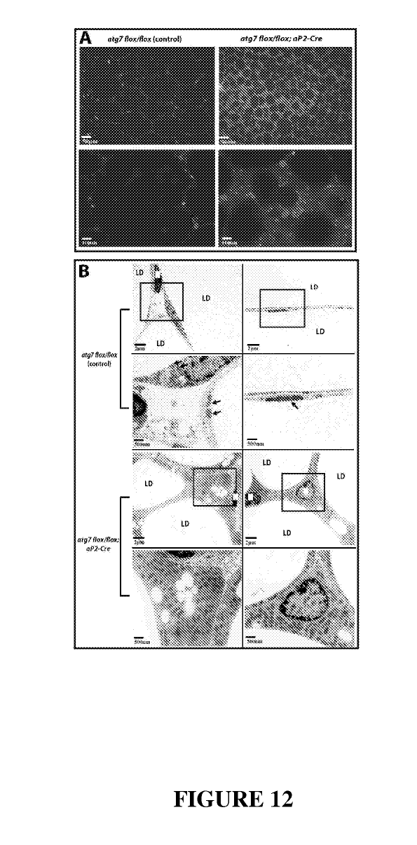

Mitochondria levels in both the atg7 knockout white adipocytes and the control atg7 wild type cells were analyzed. FIG. 12A shows immunofluorescence microscopy analysis of the cells with antibody against a mitochondrial protein, Cox II, and FIG. 12B shows electron microscopic pictures of the adipocytes. As revealed in these representative pictures, the atg7 wild type white adipocytes contained limited amount of cytoplasm and most mitochondria, if not all, were “attached” to the membrane of the lipid droplet. In contrast, the atg7 knockout adipocytes contained significant amounts of cytoplasm. In addition to the mitochondria that were associated with the lipid droplet(s), a larger fraction of mitochondria were distributed “freely” in cytoplasm. It was apparent that the atg7 knockout white adipocytes contained drastically more mitochondria than the wild type cells.

### Example 13

### Atg7 Knockout Brown Adipocytes had Smaller Lipid Droplets and Possessed Mitochondria-Saturated Cytoplasm

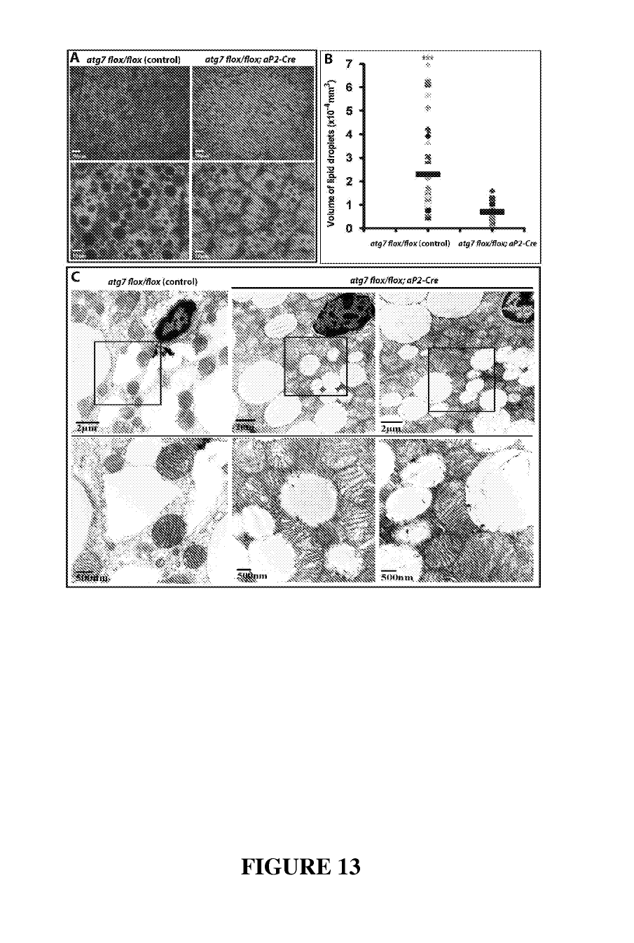

The brown fat tissue in the interscapular region of the control atg7 wild type and atg7 conditional knockout mice was analyzed. FIG. 13A shows immunofluorescence microscopy pictures with primary antibody against perilipin, which specifically labels the membrane of lipid droplets. The wild type brown fat cells contained numerous small lipid droplets. The mutant brown fat cells did not exhibit a gross morphologic difference. However, it was clear that the average diameter of the largest lipid droplets in the mutant cells was noticeably smaller than that found in the wild type cells (FIG. 13B). The brown adipocytes were further analyzed with electron microscopy (FIG. 13C). Despite the fact that brown fat cells had high mitochondria content, it was always easy to identify mitochondria-free areas and other non-lipid droplet cellular structures in the cytoplasm of the wild type cells in the electron microscopy pictures. In striking contrast, the cytoplasm of almost every single atg7 knockout brown fat cell was saturated with mitochondria. The cytoplasm was densely packed with mitochondria to such an extreme extent that except for the lipid droplets, no mitochondria-free area or other cellular structures could be easily identified. These results indicate that atg7, and by inference autophagy, is critical for maintaining normal mitochondria homeostasis in brown fat cells.

### Example 14

### Atg7−/− MEFs had Drastically Reduced Adipocyte Differentiation Efficiency

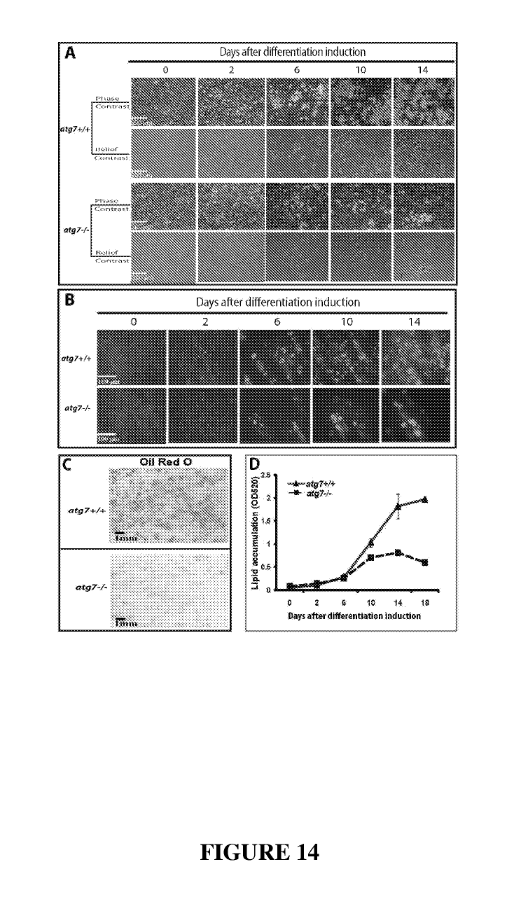

To provide further evidence that the function of atg7 is implicated in adipogenesis, an independent cellular model system was utilized to examine the impact of atg7 deletion on adipogenesis. It has been well established that the primary MEFs can be induced for differentiation into adipocytes upon hormone treatment, a process that faithfully minors many critical aspects of adipocyte differentiation in vivo. Thus, the primary MEFs provide an alternative cellular model system to study adipogenesis. The straight homozygous atg7 knockout mice are born alive but die on the first day of birth. The primary MEFs could be derived from the embryos of the atg7 straight knockout mice and their wild type littermates. These cells were induced for adipocyte differentiation under a well documented standard protocol, and the efficiency of adipocyte differentiation was compared between the wild type and atg7−/− MEFs. FIG. 14A monitored the morphological progression of differentiation with a microscope equipped with phase contrast lens as well as relief contrast lens. The relief contrast lens detects structures in three-dimensional, ideal for monitoring adipogenesis. The differentiation of the atg7−/− MEFs appeared normal in the beginning but exhibited a drastically reduced efficiency as compared to the wild type cells. Lipid accumulation is a hallmark of adipocyte differentiation. FIG. 14B shows differentiating cells fixed and stained with Bodipy 493/503, a fluorescent dye that specifically stains intracellular lipid droplets. FIGS. 14C and 14D shows cells fixed and stained with Oil Red-O, another dye that specifically stains the lipid droplets and can be extracted for spectrometry measurement. The results from these lipid accumulation assays mirrored the morphological observations. Together, these results demonstrate that deletion of atg7 drastically reduces the efficiency of adipogenesis in the primary MEFs.

### Example 15

### Adipose-Specific atg7 Knockout Mice had Reduced Plasma Concentration of Triglycerides and Cholesterol and were More Sensitive to Insulin

As described above, deletion of atg7 had a profound impact on the fat tissues. The effect of this deletion on lipid and glucose metabolism was further investigated. Fasting plasma lipid concentrations were measured in the mice at the age of 18 weeks. FIG. 15A to 15B show that the adipose-specific atg7 knockout mice had significantly reduced plasma concentrations of triglyceride and total cholesterol. Fasting glucose levels and performed glucose tolerance and insulin tolerance tests was further measured. As shown in FIG. 15E (basal levels), no significant difference in fasting plasma glucose levels was observed between the control atg7 wild type and the atg7 conditional knockout mice. The mice exhibited no significant difference in glucose tolerance test response (FIG. 15E), suggesting that the insulin secretion function of the pancreatic B cell in response to glucose elevation was normal in the adipose-specific atg7 knockout mice. However, the mutant mice exhibited significantly increased sensitivity to insulin in insulin tolerance tests (FIG. 15F), suggesting that reduction of adipogenesis in the adipose specific atg7 knockout mice had sensitized the insulin response in peripheral tissues.

### Example 16

### Adipose-Specific atg7 Knockout Mice were Resistant to High-Fat Diet Induced Obesity

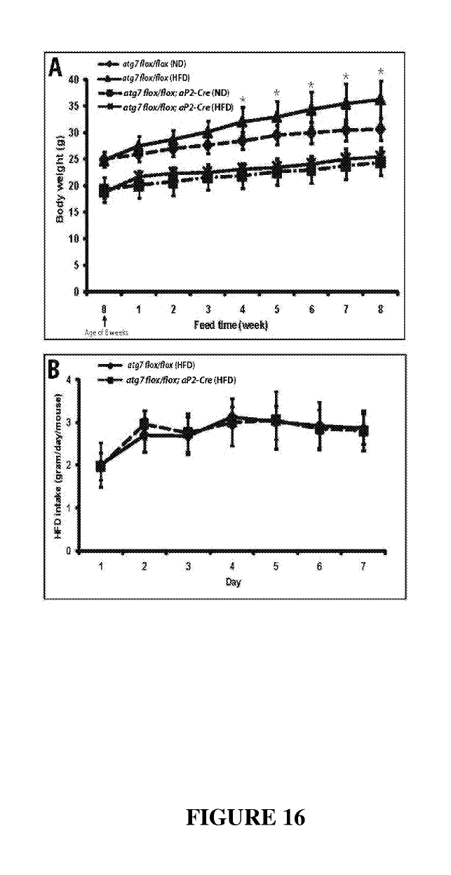

The atg7 knockout mice were lean and had drastically reduced white fat deposition. This prompted us to investigate if the adipose-specific atg7 knockout mice were more resistant to high-fat diet induced obesity. The age-matched control and atg7 conditional knockout mice were provided with a high-fat diet starting at the age of eight weeks and continued for two months. The body weight of each mouse was measured weekly. FIG. 16A shows the body weight chart while FIG. 16B shows the high-fat diet food intake rates. As expected, the wild type mice gained about 20% more body weight when fed with the high-fat diet during this two-month period as compared to mice fed with a normal diet. Strikingly, the high-fat diet caused little body weight gain in the adipose-specific atg7 conditional knockout mice. The mutant mice fed with the high-fat diet gained almost no additional weight compared to those fed a normal diet. Importantly, there was little difference between the food intake rates between the control atg7 wild type mice and the atg7 conditional knockout mice. Together, these results indicate that the adipose tissue-specific atg7 knockout mice are resistant to high-fat diet induced obesity.

1. A method for mitigating or preventing weight gain in a subject comprising, administering a therapeutically effective amount of one or more autophagy inhibitors to a subject so that differentiation of a pre-adipocyte cell into a mature adipocyte cell is inhibited.

2. The method of claim 1 wherein the autophagy inhibitor is a compound or a pharmaceutically acceptable salt thereof.

3. The method of claim 2 wherein the compound is hydroxylchloroquine.

4. The method of claim 1 wherein the autophagy inhibitor is comprised of a nucleic acid.

5. The method of claim 4 wherein the nucleic acid is selected from the group consisting of an encoding DNA enzyme, an antisense RNA, an siRNA, a shRNA, and an aptamer.

6. The method of claim 1 wherein the autophagy inhibitor is an inhibitor of autophagosome-lysosome fusion.

7. The method of claim 1 wherein the autophagy inhibitor inhibits the expression of an atg gene or the action of an ATG protein.

8. The method of claim 7 wherein the atg gene is selected from the group consisting of atg1, atg5, atg6, or atg7 and the ATG protein is selected from the group consisting of ATG1, ATG5, ATG6, and ATG7.

9. The method of claim 1 wherein the weight gain is attributed to a genetic condition.

10. The method of claim 9 wherein the genetic condition is selected from the group consisting of hypothyroidism, Cushing's syndrome, growth hormone deficiency, Prader-Willi syndrome, Bardet-Biedl syndrome, MOMO syndrome.

11. The method of claim 9 wherein the genetic condition is caused by a gene polymorphism of a leptin receptor or melanocortin receptor.

12. The method of claim 1 wherein the weight gain is attributed to smoking cessation.

13. The method of claim 1 further comprising treating one or more pathological conditions attributable to weight gain.

14. The method of claim 13 wherein the pathological conditions are selected from the group consisting of cardiovascular disease, type II diabetes, hyperlipidimia, cancers, gallbladder disease, gallstones, osteoarthritis, gout, sleep apnea and asthma.

15. A method for mitigating or preventing weight gain in a subject to whom a drug is being administered having the development of weight gain as a side effect, said method comprising:
co-administering an effective amount of an autophagy inhibitor to said subject with said drug having the development of weight gain as a side effect so that the autophagy inhibitor mitigates or prevents the weight gain side effect.

16. The method of claim 15, wherein the drug is selected from the group consisting of: lithium, Valproate, Depakote, Zyprexa, Paxil, Ergenyl, Absenor, Orfilir, Chlorpromzine, Elavil, Tofranil, Xeroxat, Cipramil, Sertralin, Zoloft, Cortisone, Prednisone, Follimin, Follinett, Neovletta, Sandomigrin, Ergenyl, and Trypizol.

17-32. (canceled)

33. A method of treating type II diabetes in a subject comprising administering a therapeutically effective amount of one or more autophagy inhibitors to a subject and increasing within the subject sensitivity to insulin.

34. The method of claim 33 wherein the autophagy inhibitor is a small molecule.

35. The method of claim 34 wherein the small molecule is hydroxylchloroquine.

36. The method of claim 34 wherein the autophagy inhibitor is comprised of a nucleic acid.

37. The method of claim 36 wherein the nucleic acid is selected from the group consisting of an encoding DNA enzyme, an antisense RNA, an siRNA, a shRNA, and an aptamer.

38. The method of claim 33 wherein the autophagy inhibitor is an inhibitor of autophagosome-lysosome fusion.

39. The method of claim 33 wherein the autophagy inhibitor inhibits the expression of an atg gene or the action of an ATG protein.

40. The method of claim 39 wherein the atg gene is selected from the group consisting of atg1, atg5, atg6, or atg7 and the ATG protein is selected from the group consisting of ATG1, ATG5, ATG6, and ATG7.
